(19) United States
(12) Patent Application Publication
(10) Pub. No.: US 2022/0062223 A1
(43) Pub. Date: Mar. 3, 2022

(54) DIHYDROMYRICETIN SPRAY-DRIED DISPENSION FORMULATIONS AND METHODS FOR FORMING THEM

(71) Applicants: The Trustees of Princeton University, Princeton, NJ (US); Cheers Health, Inc., Houston, TX (US)

(72) Inventors: Robert K. PRUD'HOMME, Princeton, NJ (US); Brooks POWELL, Houston, TX (US); Vikram PANSARE, Princeton, NJ (US); Nicholas CAGGIANO, Princeton, NJ (US); Jie FENG, Princeton, NJ (US)

(73) Assignees: The Trustees of Princeton University, Princeton, NJ (US); Cheers Health, Inc., Houston, TX (US)

(21) Appl. No.: 17/320,945

(22) PCT Filed: Nov. 8, 2019
(86) PCT No.: PCT/IB2019/001381

§ 371 Date: May 14, 2021

Related U.S. Application Data
(60) Provisional application No. 62/767,197, filed on Nov. 14, 2018.

(51) Int. Cl. A61K 31/352; A61K 9/19; A61K 9/16; A61K 45/06

(52) U.S. Cl. CPC A61K 31/352; A61K 45/06; A61K 9/1652; A61K 9/19

(57) ABSTRACT

Compositions including dihydromyricetin (DHM) and methods for forming them through spray drying.

This application claims the benefit of U.S. Provisional Application No. 62/767,197, filed Nov. 14, 2018, which is hereby incorporated by reference in its entirety.

### FIELD OF THE INVENTION

The invention pertains to compositions, processes, and methods, including spray drying and spray-dried dispersions, including dihydromyricetin.

### BACKGROUND

Alcohol is a constituent of medicines, foods, and beverages that provides both beneficial and detrimental effects on human beings. Alcohol can refer to ethyl alcohol (ethanol), which is the common form of consumable alcohol found in alcoholic beverages, e.g., such as beer, wine, and liquor. During consumption, alcohol is rapidly absorbed from the stomach and small intestine into the bloodstream, from which it can affect several organs, including the brain, heart, pancreas, and liver. Alcohol can act as a depressant to the central nervous system (CNS). For example, alcohol interferes with the brain's communication pathways, which affects brain functionality that manifests in cognitive and behavioral changes, e.g., such as a person's ability to think, focus, and move, as well as his/her mood and behavior. Alcohol can cause inflammation and damage to the liver, e.g., consistent heavy drinking can cause chronic liver problems. For example, heavy drinking can lead to steatosis (e.g., fatty liver), infection (e.g., alcoholic hepatitis), fibrosis, and cirrhosis. More commonly, even a single instance of light to moderate to heavy alcohol consumption can result in what is commonly known as an ‘alcohol hangover’. A hangover refers to an array of physical symptoms that affect a person shortly after ingesting alcohol, e.g., within hours of consumption. The symptoms of a hangover include, for example, one or more of thirst, fatigue and/or weakness, headache and/or muscle aches, dizziness/faintness, loss of appetite, poor and/or decreased sleep, nausea and/or stomach pain (e.g., which can include vomiting), and elevated heart rate. A hangover is considered to be one of the most widely experienced negative consequences of consuming ethanol.[1]

### SUMMARY OF THE INVENTION

In an embodiment of the invention, a dihydromyricetin (DHM) dosage powder includes dihydromyricetin (DHM) and a matrix material.

The matrix material can be a polymeric matrix material. The matrix material can include a linear polymer. For example, the matrix material can exclude a cyclic polymer. The matrix material can include a polymer of molecular weight of, for example, at least 5 kDa, 10 kDa, or 20 kDa. The matrix material can include cellulose or a cellulose derivative. For example, the matrix material can include hydroxypropyl methyl cellulose acetate succinate (HPMCAS), ethylcellulose, hydroxypropyl methylcellulose (HPMC), hydroxypropyl cellulose (HPC), or combinations. The matrix material can include cellulose ester, cellulose acrylate, methylcellulose, hydroxyethyl cellulose, hydroxypropyl methyl cellulose propionate succinate, hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, carboxymethyl ethylcellulose (CMEC), hydroxypropyl methylcellulose acetate phthalate (HPMCAP), hydroxypropyl methylcellulose propionate phthalate, hydroxypropyl methylcellulose acetate trimellitate (HPMCAT), hydroxypropyl methylcellulose propionate trimellitate, cellulose acetate succinate (CAS), methyl cellulose acetate succinate (MCAS), sodium carboxymethylcellulose, or combinations. The matrix material can include polyvinyl pyrrolidone (PVP) or poly(vinyl pyrrolidone-co-vinyl acetate) (PVP-VA). The matrix material can include polyethylene glycol (PEG). The matrix material can include poly(methyl methacrylate) (PMMA).

The matrix material can include a wax, polyoxyethylene-polyoxypropylene block copolymers (also referred to as poloxamers), polymethacrylates, polyoxyethylene alkyl ethers, polyoxyethylene castor oils, polycaprolactam, polycaprolactone (PCL), polylactic acid (PLA), polyglycolic acid (PGA), poly(lactic-glycolic acid) (PLGA), lipids, pullulan, dextran, hyaluronic acid, polysialic acid, chondroitin sulfate, heparin, fucoidan, pentosan polysulfate, spirulan, dextran, dextran acetate, dextran propionate, dextran succinate, dextran acetate propionate, dextran acetate succinate, dextran propionate succinate, dextran acetate propionate succinate, poly(methacrylic acid-co-methyl methacrylate) 1:1, poly(methacrylic acid-co-methyl methacrylate) 1:2, poly(methacrylic acid-co-ethyl acrylate) 1:1, a polysaccharide, polyethers, peptides, sugar oligomers, such as polydextrose and dextrans with molecular weights less than 10,000 Da, low molecular-weight oligomers of polyethylene glycols or poly amino acids or peptides, methacrylic acid copolymers, ethylene glycol-vinyl glycol copolymers, polyoxyl 40 hydrogenated castor oils, polymeric derivatives of vitamin E, N-methyl-2-pyrrolidone, cross linked polyvinyl N-pyrrolidone, a low melting point wax, such as carnauba wax, poly(propylene), poly(ethylene-co-vinyl acetate), starch, polyethoxylated sorbitan, polyoxyethylene sorbitan monooleate, polyethylene oxide, poly(ethylene oxide-co-vinyl acetate), poly(ethylene oxide-co-caprolactam), poly(ethylene oxide-co-vinyl acetate-co-caprolactam), or combinations.

The matrix material can include a sugar, sugar alcohols, organic acids, salts of organic acids, fructose, glucose, lactose, mannitol, trehalose, sucrose, raffinose, maltitol, lactitol, sorbitol, xylitol, erythritol, xylose, acorbose, melezitose, galactose, melibrose, isomaltose, natural sugar extracts, malt beet sugar, corn sugar, high-fructose corn syrup, polyols, such as glycerol, sorbitol, ethylene glycol, propylene glycol, and butanediol, amino acids and salts of amino acids, such as glycine, leucine, serine, alanine, isoleucine, tri-leucine, organic acids and salts of organic acids, such as oleic acid, citric acid, tartaric acid, edetic acid, malic acid, sodium citrate, or combinations.

The matrix material can include an amphiphilic block copolymer. For example, the amphiphilic block copolymer can be polystyrene-block-polyethylene glycol (PS-b-PEG), polylactic acid-block-polyethylene glycol (PLA-b-PEG), or poly(lactic-co-glycolic acid)-block-polyethylene glycol (PLGA-b-PEG).

The matrix material can have a glass transition temperature (Tg) of at least 70° C., 100° C., or 115° C.

The DHM dosage powder can further include a permeabilizer. For example, the permeabilizer can include capric acid, a caprate salt, and/or sodium caprate. The permeabilizer can include a permeabilizer, such as a fatty acid, a saturated fatty acid, and/or a fatty acid complexed with a cation, such as a metal cation, a metal divalent cation, a magnesium divalent cation, a calcium divalent cation, a zinc divalent cation, an iron divalent cation, a metal trivalent cation, an iron trivalent cation, a fatty acid, a fatty acid salt, a fatty acid metallic soap, or combinations.

The DHM dosage powder can further include a coactive. For example, the coactive can include an antioxidant, glutathione, L-cysteine, or combinations. For example, the coactive can include N-acetyl cysteine (NAC), Prickly Pear extract, Milk Thistle, Ginger Root, vitamin B, vitamin C, vitamin E, or combinations. The coactive can include an electrolyte and/or a sugar.

The DHM dosage powder can further include a pH buffering agent. The pH buffering agent can be an acidic pH buffering agent. The acidic pH buffering agent can include citric acid, a citrate salt, a sodium citrate, a potassium citrate, calcium citrate, and/or combinations.

In an embodiment of the invention, the DHM is not solubilized or dissolved by an aqueous solution having a pH of at most 3.5. In an embodiment of the invention, the DHM is not solubilized or dissolved by an aqueous solution having a pH of at most 2. In an embodiment of the invention, the DHM is solubilized or dissolved by an aqueous solution having a pH of at least 5.5. In an embodiment of the invention, the DHM is solubilized or dissolved by water or an aqueous solution having a pH of at least 7.

In the DHM dosage powder, the DHM can include at least 5 wt %, at least 20 wt %, at least 40 wt %, or at least 55 wt % of the powder.

In the DHM dosage powder, the crystallinity of the DHM can be at most 20%, at most 10%, at most 5%, or at most 2%, or the DHM can be amorphous.

The DHM dosage powder of any one of claims 1 through 42, wherein the DHM dosage powder is molecularly dispersed, uniformly dispersed, or homogeneous.

In the DHM dosage powder, the DHM and the matrix material can be homogeneously, uniformly, or molecularly dispersed in each other, and the crystallinity of the DHM can be at most 10% or at most 5%, or the DHM can be amorphous.

In the DHM dosage powder, the size of 80 wt % of particles in the powder can be at least 5 μm or at least 10 μm.

In an embodiment of the invention, a dosage form includes the DHM dosage powder and an enteric coating that encapsulates the DHM dosage powder. For example, the enteric coating can be a polymeric coating or a methacrylate copolymer coating. The dosage form can be a capsule, tablet, or pill.

In an embodiment of the invention, a dosage form includes the DHM dosage powder and an aqueous liquid, and the DHM dosage powder is mixed with or suspended in the liquid.

In an embodiment of the invention, a dosage form includes the DHM dosage powder and a gel, and the DHM dosage powder is mixed with or suspended in the gel.

In an embodiment of the invention, in the DHM dosage powder, the matrix material includes the polymer hydroxypropyl methyl cellulose acetate succinate (HPMCAS), the DHM forms at least 10 wt % of the powder, and the crystallinity of the DHM is at most 10% or at most 5%, or the DMH is amorphous. The DHM and the matrix material can be homogeneously or uniformly dispersed in each other.

In an embodiment of the invention, in the DHM dosage powder, the matrix material is hydroxypropyl methyl cellulose acetate succinate (HPMCAS), the DHM includes at least 20 wt % of the powder, and the crystallinity of the DHM is at most 10% or at most 5%, or the DMH is amorphous. The DHM and the matrix material can be homogeneously or uniformly dispersed in each other.

In an embodiment of the invention, in the DHM dosage powder, the matrix material is hydroxypropyl methyl cellulose acetate succinate (HPMCAS), the DHM includes at least 40 wt % of the powder, and the crystallinity of the DHM is at most 10% or at most 5%, or the DMH is amorphous. The DHM and the matrix material can be homogeneously or uniformly dispersed in each other.

In an embodiment of the invention, mixing of the DHM dosage powder with a solvent results in a concentration of DHM dissolved in the solvent that is at least 20% greater than the equilibrium concentration of crystalline DHM dissolved in the solvent. The solvent can be an aqueous solvent, can have a pH in the range of 4.5 to 7.0, can include sodium at a concentration of from 100 mM to 400 mM, and/or can include a surfactant, a nonionic surfactant, a polysorbate, and/or Polysorbate 20 at a concentration of from 0.01 wt %, 0.02 wt %, 0.05 wt %, 0.1 wt %, 0.2 wt %, or 0.5 wt % to 0.05 wt %, 0.1 wt %, 0.2 wt %, 0.5 wt %, 1 wt %, or 2 wt %. For example, the solvent can include a surfactant, a nonionic surfactant, a polysorbate, and/or Polysorbate 20at a concentration of from 0.05 wt %, 0.1 wt %, 0.2 wt %, or 0.5 wt % to 0.2 wt %, 0.5 wt %, 1 wt %, or 1.5 wt %. For example, the solvent can include fed state simulated intestinal fluid (FeSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %. For example, the solvent can include fasted state simulated intestinal fluid (FaSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %.

In an embodiment of the invention, mixing of the DHM dosage powder with a solvent results in a concentration of DHM dissolved in the solvent that is at least 50% greater than the equilibrium concentration of crystalline DHM dissolved in the solvent. The solvent can be an aqueous solvent, can have a pH in the range of 4.5 to 7.0, can include sodium at a concentration of from 100 mM to 400 mM, and/or can include a surfactant, a nonionic surfactant, a polysorbate, and/or Polysorbate 20 at a concentration of from 0.01 wt %, 0.02 wt %, 0.05 wt %, 0.1 wt %, 0.2 wt %, or 0.5 wt % to 0.05 wt %, 0.1 wt %, 0.2 wt %, 0.5 wt %, 1 wt %, or 2 wt %. For example, the solvent can include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt %. For example, the solvent can include fed state simulated intestinal fluid (FeSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %. For example, the solvent can include fasted state simulated intestinal fluid (FaSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %.

In an embodiment of the invention, mixing of the DHM dosage powder with a solvent results in a concentration of DHM dissolved in the solvent that is at least 70% greater than the equilibrium concentration of crystalline DHM dissolved in the solvent. The solvent can be an aqueous solvent, can have a pH in the range of 4.5 to 7.0, can include sodium at a concentration of from 100 mM to 400 mM, and/or can include a surfactant, a nonionic surfactant, a polysorbate, and/or Polysorbate 20 at a concentration of from 0.01 wt %, 0.02 wt %, 0.05 wt %, 0.1 wt %, 0.2 wt %, or 0.5 wt % to 0.05 wt %, 0.1 wt %, 0.2 wt %, 0.5 wt %, 1 wt %, or 2 wt %. For example, the solvent can include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt %. For example, the solvent can include fed state simulated intestinal fluid (FeSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %. For example, the solvent can include fasted state simulated intestinal fluid (FaSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %.

In an embodiment of the invention, mixing of the DHM dosage powder with a solvent results in a concentration of DHM dissolved in the solvent that is at least 100% greater than the equilibrium concentration of crystalline DHM dissolved in the solvent. The solvent can be an aqueous solvent, can have a pH in the range of 4.5 to 7.0, can include sodium at a concentration of from 100 mM to 400 mM, and/or can include a surfactant, a nonionic surfactant, a polysorbate, and/or Polysorbate 20 at a concentration of from 0.01 wt %, 0.02 wt %, 0.05 wt %, 0.1 wt %, 0.2 wt %, or 0.5 wt % to 0.05 wt %, 0.1 wt %, 0.2 wt %, 0.5 wt %, 1 wt %, or 2 wt %. For example, the solvent can include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt %. For example, the solvent can include fed state simulated intestinal fluid (FeSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %. For example, the solvent can include fasted state simulated intestinal fluid (FaSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %.

In an embodiment of the invention, mixing of the DHM dosage powder with a solvent results in a concentration of DHM dissolved in the solvent that is at least 200% greater than the equilibrium concentration of crystalline DHM dissolved in the solvent. The solvent can be an aqueous solvent, can have a pH in the range of 4.5 to 7.0, can include sodium at a concentration of from 100 mM to 400 mM, and/or can include a surfactant, a nonionic surfactant, a polysorbate, and/or Polysorbate 20 at a concentration of from 0.01 wt %, 0.02 wt %, 0.05 wt %, 0.1 wt %, 0.2 wt %, or 0.5 wt % to 0.05 wt %, 0.1 wt %, 0.2 wt %, 0.5 wt %, 1 wt %, or 2 wt %. For example, the solvent can include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt %. For example, the solvent can include fed state simulated intestinal fluid (FeSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %. For example, the solvent can include fasted state simulated intestinal fluid (FaSSIF), which can further include a surfactant, a nonionic surfactant, a polysorbate, and/or Polysorbate 20 at a concentration of from 0.05 wt %, 0.1 wt %, 0.2 wt %, or 0.5 wt % to 0.2 wt %, 0.5 wt %, 1 wt %, or 1.5 wt % or about 1 wt %.

A method for forming the dihydromyricetin (DHM) dosage powder or the dosage form includes dissolving the dihydromyricetin (DHM) and the matrix material in a solvent to form a spray solution; forcing the spray solution through an atomizer to atomize the spray solution; and allowing the solvent to volatilize from the atomized spray solution to form the DHM dosage powder. The spray solution can enter a drying chamber after being forced through the atomizer. The volatilization of the solvent can cause the polymer matrix material to gel without the DHM or matrix material phase separating from the atomized spray solution. The solvent can include acetone, tetrahydrofuran (THF), methanol, ethanol, dimethylsulfoxide (DMSO), and/or water. The solvent can include an alcohol, n-propanol, iso-propanol, butanol, a ketones, acetone, methyl ethyl ketone (MEK), methyl iso-butyl ketone, an ester, ethyl acetate, propylacetate, acetonitrile, methylene chloride, toluene, a cyclic ether, 1,1,1-trichloroethane, a lower volatility solvent, dimethyl acetamido, or combinations. The temperature of the drying chamber can be at least 5° C., 10° C., or, 20° C. greater than a boiling point temperature of the solvent.

A method for forming the dihydromyricetin (DHM) dosage powder includes dissolving the dihydromyricetin (DHM) and hydroxypropylmethylcellulose acetate succinate (HPMCAS) as the matrix material in acetone at a total solids concentration of at least 2%, 5% or 10% to form a spray solution, with the weight ratio of DHM to HPMCAS being at least 5:95, forcing the spray solution through an atomizer to atomize the spray solution, and allowing the solvent to volatilize from the atomized spray solution to form the dihydromyricetin (DHM) dosage powder. The weight ratio of DHM to HPMCAS can be at least 10:90, at least 20:80, or at least 40:60.

A method for forming the dihydromyricetin (DHM) dosage powder includes dissolving the dihydromyricetin (DHM) and the hydroxypropylmethylcellulose acetate succinate (HPMCAS) in acetone at a total solids concentration of at least 10% to form a spray solution, with the weight ratio of DHM to HPMCAS being at least 5:95, forcing the spray solution through an atomizer to atomize the spray solution, and allowing the solvent to volatilize from the atomized spray solution to form the dihydromyricetin (DHM) dosage powder. The weight ratio of DHM to HPMCAS can be at least 10:90, at least 20:80, or at least 40:60.

A method for forming the dihydromyricetin (DHM) dosage powder or the dosage form includes dissolving the dihydromyricetin (DHM) and the matrix material in a solvent to form a solution, and then allowing the solvent to volatilize or evaporate from the solution to form the DHM dosage powder or the DHM and matrix material in another solid form.

The dihydromyricetin (DHM) dosage powder or the dosage form can be used as a medicament. For example, the dihydromyricetin (DHM) dosage powder or the dosage form can be used in reducing hangover symptoms, preventing an alcohol use disorder, preventing alcoholism, treating an alcohol use disorder, treating alcoholism, treating an alcohol overdose, increasing antioxidant capacity, neuroprotection, preventing Alzheimer's disease, treating Alzheimer's disease, inhibiting inflammation, protecting the kidney, protecting the liver, preventing or treating cancer, ameliorating a metabolic disorder, preventing diabetes, treating diabetes, and/or treating a bacterial infection.

The dihydromyricetin (DHM) dosage powder can be used in the manufacture of a medicament, such as for reducing hangover symptoms, preventing an alcohol use disorder, preventing alcoholism, treating an alcohol use disorder, treating alcoholism, treating an alcohol overdose, neuroprotection, preventing Alzheimer's disease, treating Alzheimer's disease, ameliorating a metabolic disorder, preventing diabetes, treating diabetes, increasing antioxidant capacity, inhibiting inflammation, protecting the kidney, protecting the liver, preventing and/or treating cancer, and/or treating a bacterial infection.

### BRIEF DESCRIPTION OF THE DRAWINGS

FIG. 1 presents Powder X-Ray Diffraction (PXRD) traces for the starting crystalline DHM material and a spray dried dispersion (SDD) formulation including DHM (20% DHM/80% HPMCAS).

FIGS. 2A and 2B are micrographs of crystalline DHM (FIG. 2A) and the SDD formulation (20% DHM/80% HPMCAS) (FIG. 2B), obtained by Scanning Electron Microscopy (SEM).

FIG. 3 presents in vitro release data of the starting crystalline DHM material and the SDD formulation (20% DHM/80% HPMCAS) in fed state simulated intestinal fluid (FeSSIF) over a 6 hour time period with the following sampling timepoints: 1, 5, 10, 15, 30, 60, 120, and 360 minutes.

FIG. 4 presents in vitro release data of the starting crystalline DHM material and the SDD formulation (40% DHM/60% HPMCAS) in fed state simulated intestinal fluid (FeSSIF) including 1% v/v Tween 20 (Polysorbate 20) over a 6 hour time period with the following sampling timepoints: 1, 5, 10, 15, 30, 60, 120, and 360 minutes.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are hereby incorporated by reference in their entirety as if each had been individually incorporated.

An aspect of the present invention includes a method to improve the bioavailability of the molecule dihydromyricetin (DHM) through the process known as spray drying (SD) to form a spray dried dispersion (SDD). This method can include processing by SD of a combination of materials including DHM, additional beneficial molecules (e.g., co-actives), polymeric excipients, permeabilizers, and solvents. The final form of the product may comprise powders, granules, or tablets to be used in further formulations.

The present invention can provide a spray drying method and resultant formulation including a beneficial amount of DHM, additional beneficial molecules, polymeric excipients, and permeabilizers and solvents. Improvements in bioavailability and pharmacokinetic parameters of DHM can be associated with this formulation and method.

In an embodiment, the formulation may be processed further in forms beyond powders, granules, and tablets for administration by various routes either by self-administration or administration by any number of routes known to a skilled artisan. In some embodiments, the formulation may be especially well suited to oral administration routes.

CN1293825C (2002) discusses extracting DHM from a plant source and preparing by spray drying or spray freeze drying the DHM extract for use as an antioxidant in food products.[3] CN104666293A (2015) discusses using cyclodextrin (CD) to form DHM-CD inclusion complexes, which are then prepared for administration through the use of a spray drying process. [4] CN105796512A (2016) discusses preparing DHM in solid lipid granules by freeze drying.[5] Yao (2007) discusses the spray drying of DHM with gum Arabic and/or maltodextrin.[6] Curatolo et al. (2001) discusses the preparation of low-solubility drugs as spray-dried dispersions using hydroxypropylmethylcellulose acetate succinate (HPMCAS) as a matrix material in the spray drying process. Beyerinck et al. (2003) and Beyerinck et al. (2003) discusses modifications to spray-drying process equipment, including the use of a pressure nozzle and diffuser plate, to improve the fluid dynamics within the spray dry chamber and create more homogeneous solid dispersions of drugs in concentration enhancing polymers. Perlman et al. (2003) discusses to the use of lipophilic microphase forming materials in solid dispersions of drug. Babcock et al. (2003) relates to the preparation of spray dried dispersions with a drug and a polymer. Beyerinck et al. (2004) discusses processes and equipment used to produce spray dried dispersions of drugs within polymeric excipients/matrices. Smithey et al. (2004) discusses solid compositions of drugs with poloxamer excipients. Crew (2004) discusses solid compositions with poloxamers and an additional cellulosic polymer.

Bulk solvent evaporation, for example, evaporating solvent from a solution over time, optionally with heating of the solution or placing the solution under a partial or full vacuum to accelerate the evaporation, can be used to form a powder or solid containing a mixed drug, polymer, and/or other components.

In freeze drying, a solution or suspension is frozen and then placed under vacuum; the ice then sublimes over time to leave behind the pure compound or suspension. The term spray drying describes the preparation of a compound by dissolution and subsequent drying in a spray drying chamber with hot drying gases to produce a powder of the pure compound. The formation of spray dried dispersions (SDDs) in an embodiment of the present invention involves the dispersion of an active compound or compounds within a matrix material, for example, a polymeric and/or cellulosic material.

### DHM

Dihydromyricetin (DHM), a flavonoid compound isolated from the Hovenia plant, can “sober-up” rats inebriated with alcohol[2], prevent predisposed rats from becoming alcoholics[2], return alcoholic rats to baseline levels of alcohol consumption[2], reduce hangover symptoms[2], and prevent fetal alcohol spectrum disorders in the offspring of rats exposed to significant amounts alcohol during pregnancy.[2] DHM can be dissolved in a solvent, such as dimethylsulfoxide (DMSO). DHM can be complexed with a metal, such as a divalent alkali earth metal, divalent magnesium (Mg(II), Mg⁺²), a divalent transition metal, divalent iron (Fe(II), Fe⁺²), divalent copper (Cu(II), Cu⁺²), a trivalent transition metal, or trivalent iron (Fe(III), Fe⁺²).

DHM has unique physicochemical properties including low solubility, high hydroxyl functional group content, and unknown thermal stability, rendering the processing of DHM and other flavonoids to form spray-dried dispersions for therapeutic administration and to improve their dissolution kinetics and bioavailability difficult.

DHM demonstrates the pharmacological properties expected to underlie successful medical treatment of alcohol use disorders (AUDs)[7-9]. Given limited available pharmacotherapies for AUDs and these being limited by low patient compliance, because of the adverse effects they cause, therapies for the treatment of AUDs should be advanced, e.g., through DHM therapeutic strategies.[10]

In addition to DHMs potential for the treatment of AUDs, which, without being bound by theory, may be achieved through DHM's inhibiting the effect of alcohol on GABAA receptors (GABAARs) in the brain, DHM and the Hovenia plant it is isolated from have shown efficacy in mitigating liver injuries[11-13], decreasing alcohol and acetaldehyde concentrations in the blood via enhancing ADH and ALDH activity[14, 15], and eliminating alcohol-induced excessive free radicals.[16] DHM has been observed to have oxidative stress-mediating activity, i.e., increase antioxidant capacity for scavenging reactive oxygen species, which may result in neuroprotective, nephroprotective (kidney protecting), and hepatoprotective (liver protecting) effects, which may ameliorate, for example, the effects of hypobaric hypoxia, side effects of the chemotherapeutic agent cisplatin, and detrimental effects of ethanol. DHM may have a neuroprotective role in Alzheimer's and Parkinson's diseases. DHM can also inhibit inflammation. DHM can also have anticancer activity and regulate cell proliferation and apoptosis. DHM can mediate metabolism, and may be useful in ameliorating certain metabolic disorders, such as diabetes, weight gain, hyperlipidemia, and atherosclerosis. DHM exhibits antibacterial activity (Li, H. et al., “The Versatile Effects of Dihydromyricetin in Health”, EvidenceBased Complementary & Alternative Medicine 2017, Art. ID 1 053617).

A DHM formulation designed to reduce alcohol's negative effects when taken after alcohol consumption is covered under U.S. Pat. No. 9,603,830 B2 (granted on Mar. 28, 2017) and is sold in the US under the brand name Thrive+®.

Despite promising results in rats, one challenge in translating DHM's efficacy to humans in a commercially viable way is DHM's oral bioavailability of less than 5%[17]. DHM is a BCS class IV drug limited by having the properties of both low solubility and permeability. In the context of successfully commercialized drugs, DHM requires large doses for efficacy. Because DHM is a naturally occurring organic compound isolated from an herb, a DHM formulation can be classified as a food (or dietary supplement) under the Dietary Products designation.

This invention addresses the problem of poor bioavailability and stability of DHM through the use of spray dried dispersion (SDD) technology. By dispersing DHM within a set of excipients using SDD, e.g., excipients which are chiefly polymeric, DHM may exhibit enhanced dissolution and release kinetics, longer sustained release, higher concentrations, and improved stability with respect to low pH gastric juices and enzymes, which can cause degradation and quenching of DHM activity, than when administered in a pure form. Furthermore, the DHM SDD formulation may possess improved ability to penetrate intestinal barriers, to allow DHM to reach the bloodstream more effectively and efficiently.

### Spray Dried Dispersions

25-30% of pharmaceutical compounds in early development have been estimated to have low solubility and/or bioavailability, reducing the potential efficacy of the drug and often requiring larger doses to achieve therapeutic effects. Such larger doses can have detrimental side effects, and if larger doses must be administered to achieve a therapeutic effect, the therapeutic window (the difference in the dosage that results in a toxic or detrimental effect and the dosage that results in a therapeutic effect) can be narrowed. Because many of these drugs have the potential to be both safe and efficacious, the delivery of these compounds should be improved.

In an embodiment of the present invention, spray drying is used to improve the solubility and/or bioavailability of DHM. The term Spray Dried Dispersion (SDD) denotes not just the process of making a dry powder from spraying and drying a solution or dispersion. The term, as used in this text, also includes the use of excipients, e.g., polymers, that have specific associations with the active compound being spray dried, so that the spray dried drug dispersion (SDDs) can be used to improve the bioavailability of DHM and poorly water soluble drugs.

DHM or a drug can be dissolved in a solvent along with a matrix forming material. The solution (spray solution) can be pumped to and through a spray nozzle (atomizer) where the mixture is rapidly atomized, and then dried by hot gases to form a free flowing powder. Such an SDD can accomplish the following objectives: (1) enhance the oral absorption of poorly water-soluble compounds by attaining and sustaining a supersaturated concentration of drug in the gastrointestinal (GI) fluid; (2) provide a physically stable drug form (avoiding crystallization or phase separation of amorphous drug) that enables processing of the dispersion into solid dosage forms for shipment and usage; (3) provide a solid drug form that can be manufactured via a reproducible, controllable, and scalable process; and (4) provide a technology that is applicable to structurally diverse insoluble compounds across a wide range of physicochemical properties.

A DHM spray-dried dispersion dosage form that is an embodiment of the invention can include, but is not limited to, the active ingredient DHM and additional beneficial active molecules (co-actives), permeability enhancers, excipients (including matrix materials), and/or solvents. The DHM spray-dried dispersion dosage form can be a powder. In an embodiment, the DHM spray-dried dispersion dosage form includes dihydromyricetin (DHM) and a coactive, such as L-cysteine, N-acetyl cysteine (NAC), Prickly Pear extract, Milk Thistle, ginger root, vitamin B, vitamin C, vitamin E, an electrolyte, and/or a sugar.

### Permeabilizers

A permeability-enhancer or permeabilizer is an agent that enhances the permeation of a drug compound through the epithelial cell layer in the gastrointestinal (GI) tract and, hence, enhances the amount of drug entering the bloodstream. Permeability-enhancers have been reviewed by Aungst and Whitehead[18-21]. The list of agents presented by Aungst in Table I and Whitehead in Table I are incorporated into this patent in their entirety.

Examples of permeability-enhancers are fatty acids, a saturated fatty acid, capric acid, a caprate salt, sodium caprate, a fatty acid complexed with a cation, such as a metal cation, magnesium, calcium, zinc divalent cation (Zn(II), Zn⁺²), iron divalent cation (Fe(II), Fe⁺²), iron trivalent cation (Fe(III), Fe⁺³), or combinations. For example, capric acid and its salts are permeabilizers that are currently clinically approved for use in an ampicillin suppository. The caprates and other long-chain saturated fatty acids and their salts can be incorporated into the spray drying process. Their hydrophobicity can be enhanced by complexing them, for example, with divalent cations such as those of magnesium, calcium, or zinc, divalent iron, or trivalent iron. Permeabilizers are optional additions to the formulation. When they are used, the mass ratios of permeabilizer to DHM in the spray dried dispersion produced can range from 1:100 to 100:1.

### Excipients and Matrix Materials

Excipients and matrix materials are defined as materials which aid in the formulation, stability, and/or release characteristics of the active molecule DHM. For example, homopolymers, copolymers, and amphiphilic copolymers can be used as excipients and matrix materials. The matrix material can constitute from 0.1 wt % to 99 wt % of the combined mass of the active agent(s) and excipients by weight of the final solid form. When it is desirable for the matrix material to prevent aggregation of the active domains into larger aggregates, the matrix material can constitute more than 20% or more than 40% of the combined mass of the active agent(s) and matrix material. For example, the active agent can constitute at least 1, 2, 5, 10, 15, 20, 25, 30, 40, 50, 60, 70, 80, 90, or 95 wt % of the combined mass of the active agent(s) and matrix material. For example, the active agent can constitute at most 2, 5, 10, 15, 20, 25, 30, 40, 50, 60, 70, 80, 90, 95, or 98 wt % of the combined mass of the active agent(s) and matrix material. In some cases, the excipients or matrix materials may be selected from materials having low molecular weight, for example, for inhalation applications.

Exemplary excipients and matrix materials include starches, waxes, polyvinyl pyrrolidone (PVP), polyethyleneoxide (PEO), polyethylene glycol (PEG), polyvinyl pyrrolidone-co-vinyl acetate) (PVP-VA), polyoxyethylene-polyoxypropylene block copolymers (also referred to as poloxamers), polymethacrylates, polyoxyethylene alkyl ethers, polyoxyethylene castor oils, polycaprolactam, polycaprolactone (PCL), polylactic acid (PLA), polyglycolic acid (PGA), poly(lactic-glycolic)acid (PLGA), lipids, cellulose, pullulan, dextran, maltodextrin, hyaluronic acid, polysialic acid, chondroitin sulfate, heparin, fucoidan, pentosan polysulfate, spirulan, hydroxypropyl cellulose (HPC), hydroxypropyl methyl cellulose acetate succinate (HPMCAS), hydroxypropyl methyl cellulose propionate succinate, hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, carboxymethyl ethylcellulose (CMEC), hydroxypropyl methylcellulose (HPMC), hydroxypropyl methylcellulose acetate phthalate (HPMCAP), hydroxypropyl methylcellulose propionate phthalate, hydroxypropyl methylcellulose acetate trimellitate (HPMCAT), hydroxypropyl methylcellulose propionate trimellitate, cellulose acetate succinate (CAS), methyl cellulose acetate succinate (MCAS), dextran, dextran acetate, dextran propionate, dextran succinate, dextran acetate propionate, dextran acetate succinate, dextran propionate succinate, dextran acetate propionate succinate, poly(methacrylic acid-co-methyl methacrylate) 1:1 (e.g., Eudragit® L100, Evonik Industries AG), poly(methacrylic acid-co-methyl methacrylate) 1:2 (e.g., Eudragit® S100), poly(methacrylic acid-co-ethyl acrylate) 1:1 (e.g., Eudragit® L100-55), and mixtures thereof.

Examples of excipients and matrix materials include sugars, sugar alcohols, polyols (as exemplified above), polyethers (as exemplified above), cellulosic polymers (as exemplified above), amino acids, salts of amino acids, peptides, organic acids, salts of organic acids, and mixtures thereof. Specific examples of sugars and sugar alcohols include, but are not limited to fructose, glucose, lactose, mannitol, trehalose, sucrose, raffinose, maltitol, lactitol, sorbitol, xylitol, erythritol, xylose, acorbose, melezitose, galactose, melibrose, isomaltose. Natural sugar extracts, including, but not limited to malt beet sugar, corn sugar, high-fructose corn syrup, and sugar oligomers, such as polydextrose and dextrans with molecular weights less than 10,000 Daltons. Polyols such as glycerol, sorbitol, ethylene glycol, propylene glycol, butanediol, and other oligomers. Amino acids and salts of amino acids, such as glycine, leucine, serine, alanine, isoleucine, tri-leucine. Organic acids and salts of organic acids, such as oleic acid, citric acid, tartaric acid, edetic acid, malic acid, sodium citrate, and mixtures thereof. Low molecular-weight oligomers are suitable including polyethylene glycols, poly amino acids or peptides and copolymers such as polyethylene glycol/polypropylene glycol copolymers, poloxamers, and mixtures thereof. In one embodiment, the matrix material is selected from fructose, glucose, lactose, mannitol, trehalose, sucrose, raffinose, maltitol, lactitol, sorbitol, xylitol, erythritol, xylose, acorbose, melezitose, galactose, melibrose, isomaltose, malt beet sugar, corn sugar, high-fructose corn syrup, polydextrose, and dextrans with molecular weights less than 10,000 Daltons, glycerol, ethylene glycol, propylene glycol, butanediol, glycine, leucine, serine, alanine, isoleucine, tri-leucine, oleic acid, citric acid, tartaric acid, edetic acid, malic acid, sodium citrate, low molecular-weight polyethylene glycols, poly amino acids, polyethylene glycol/polypropylene glycol copolymers, poloxamers, and mixtures thereof. In another embodiment, matrix material is selected from fructose, glucose, lactose, mannitol, trehalose, sucrose, raffinose, maltitol, lactitol, sorbitol, xylitol, erythritol, xylose, acorbose, melezitose, galactose, melibrose, isomaltose, malt beet sugar, corn sugar, high-fructose corn syrup, polydextrose, and dextrans with molecular weights less than 10,000 Daltons. In still another embodiment, the matrix material is selected from glycine, leucine, serine, alanine, isoleucine, tri-leucine, oleic acid, citric acid, tartaric acid, edetic acid, malic acid, sodium citrate, and mixtures thereof. When a crystalline form, the matrix material is preferably selected from the group consisting of lactose, mannitol, trehalose, and mixtures thereof.

In an embodiment, the matrix material includes components with a molecular weight of less than 1,000,000 Daltons (Da), less than 100,000 Daltons, less than 10,000 Daltons, less than 5000 Daltons, or less than 2000 Daltons.

The matrix material can include a polymer. A polymer is formed of several monomer units bound to each other. For example, a polymer can be a linear polymer, a branched polymer, or a cyclic polymer. In a cyclic polymer, a set of monomers can be bound to each other to form a ring. In a noncyclic polymer, there is no set of monomers that are bound to each other to form a ring (although atoms within a given monomer unit of the polymer still may be in a ring structure, e.g., a cyclopentyl, furan, furanose, cyclohexyl, pyran, pyranose, benzene, or saccharide structure). For example, cyclodextrin is a cyclic polysaccharide. By contrast, cellulose is a linear polysaccharide formed of several hundred to many thousands of D-glucose monomers. Gum arabic includes arabinogalactan, formed of arabinose and galactose monomers.

Certain polymeric excipients and matrix materials marketed under trade names by manufacturers may include:

Povidones, copovidones, methacrylic acid copolymers, ethylene glycol-vinyl glycol copolymers, Poloxamer 407, Poloxamer 188, poly ethylene glycols, polyoxyl 40 hydrogenated castor oils, and polymeric derivatives of vitamin E marketed by BASF under trade names SOLUPLUS, KOLLIDON VA 64, KOLLIDON 12 PF, KOLLIDON 17 PF, KOLLIDON 30, KOLLIDON 90 F, KOLLIDON SR, KOLLICOAT MAE 100P, KOLLICOAT IR, KOLLICOAT PROTECT, KOLLIPHOR P 407, KOLLIPHOR P407 MICRO, KOLLIPHOR P188, KOLLIPHOR P188 MICRO, KOLLISOLV PEG, KOLLIPHOR RH 40, AND KOLLIPHOR TPGS, marketed by BASF.

Polymers with trade names ETHOCEL, POLYOX, and AFFINISOL marketed by the Dow Chemical Company.

Polymers with trade names EUDRAGIT (methacrylates), and RESOMER, marketed by Evonik Corporation.

Polymers with trade names AquaSolve hypromellose acetate succinate, Aqualon ethylcellulose, Aqualon sodium carboxymethylcellulose, Aquarius control film coating systems, Aquarius prime film coating systems, Aquarius protect film coating systems, Aquarius film coating systems, Aquarius preferred film coating systems, Benecel methylcellulose and hypromellose, Blanose sodium carboxymethylcellulose, CAVAMAX native cyclodextrins, Cavitron cyclodextrin, CAVASOL cyclodextrin, Klucel hydroxypropylcellulose, Natrosol hydroxyethylcellulose, Pharmasolve N-methyl-2-pyrrolidone, Plasdone S-630 copovidone, Plasdone povidone, and Polyplasdone crospovidone marketed by Ashland Global Holdings Inc.

The foregoing lists of materials are not intended to indicate that all of these materials are equivalent and/or equally suitable.

The polymer matrix material can have a glass transition temperature (Tg) of at least 50° C., 60° C., 70° C., 80° C., 90° C., 100° C., 110° C., 115° C., 120° C., 125° C., 130° C., 150° C., 175° C., 200° C., or 250° C. For example, hydroxypropyl methyl cellulose acetate succinate (HPMCAS) has a glass transition temperature (Tg) of about 120° C.

The polymer matrix material may be selected to adjust the formulation's release profile, e.g., to adjust the rate at and duration of time over which the formulation releases an active pharmaceutical ingredient (API), such as DHM.

In an embodiment, polymers, such as one or more of those listed above, may also be incorporated as enteric coatings which coat a final tablet form of a DHM spray-dried dispersion and provide additional stability or sustained release benefits. For example, including an enteric coating in the formulation may alter the formulation's release profile, e.g., may alter the rate at and duration of time over which the formulation releases an active pharmaceutical ingredient (API), such as DHM.

### Composition of the Components

In some embodiments, DHM constitutes at least 0.1 wt %, 1 wt %, 2 wt %, 3 wt %, 5 wt %, 7 wt %, 10 wt %, 15 wt %, 20 wt %, 25 wt %, 30 wt %, 35 wt %, 40 wt %, 45 wt %, 50 wt %, 55 wt %, 60 wt %, 70 wt %, 80 wt %, 90 wt %, 95 wt %, 98 wt %, or 99 wt % of the spray-dried powder, relative to all other excipients and matrix materials.

In some embodiments, the concentration of all other components, and particularly excipients and matrix materials, in the formulation may range from 0.001 wt % to 0.01 wt %, or from 0.01 wt % to 0.1 wt %, or from 0.1 wt % to 1 wt %, or from 1 wt % to 10 wt %, or from 10 wt % to 99.9 wt %, depending on the desired release profile, the pharmacological activity and toxicity of the therapeutic compound, such as DHM and any coactive, and other considerations.

Spray Dried Dispersion Process

A spray-drying process according to the present invention can form compositions comprising homogeneous solid dispersions of flavonoids, such as DHM, and excipients for the purpose of improving the delivery of flavonoids in animals and humans. Such compositions can have improved bioavailability. In an embodiment of a process according to the invention, homogeneous, solid dispersions are formed by first dissolving the DHM and excipients (including polymeric matrix materials and any other excipients and permeabilizers) in a solvent to form a spray solution. The solvent is then rapidly removed to form a solid dispersion.

The use of spray drying in the preparation of dosage forms to effectively deliver DHM has several advantages. SDD is scalable, because of its continuous nature, and can produce a pharmaceutically-appropriate powder dosage form that can be further processed into tablets or other morphologies having desired surface characteristics.

Spray drying allows for DHM to be formulated with a wide variety of excipients, such as matrix materials, to allow for maximum effectiveness for various administration routes. The spray drying process can trap active molecules, such as DHM, in a high-energy amorphous state to improve bioavailability and sustained concentrations in the bloodstream.

The term spray drying broadly refers to processes involving breaking up liquid mixtures containing a dissolved or dispersed solid into small droplets (atomization) and rapidly removing solvent from the droplets in a container (spray drying chamber or drying chamber) where there is a strong driving force (e.g., an elevated temperature) for the evaporation of solvent, so that solid particles are formed.

In an embodiment, the spray-drying system includes tanks or hoppers for drug, excipients, and solvent. The system includes a tank for mixing the spray solution using a mixer. The spray solution can include the dissolved drug(s), such as DHM and other beneficial molecules (coactives), and excipients in a solvent. An optional solvent tank may be employed to aid in processing. The tank is connected via a feedline having a pump to an atomizer located at the top of a drying chamber. The pump forces the spray solution through the atomizer under pressure. The atomizer breaks the spray solution up into fine droplets in the drying chamber. A drying gas, such as nitrogen, is also introduced through an inlet or a gas disperser into the chamber. The solvent evaporates from the droplets within the chamber, forming solid dispersion particles of drug and excipient(s). The solid dispersion particles and exhaust drying gas (the now cooler drying gas and evaporated solvent) exit the drying chamber out of an outlet at the bottom of the drying chamber. The solid dispersion particles may be separated from the exhaust gas by means of a cyclone or another collection device.

Spray Solution and Drying Conditions

The spray solution and drying conditions can be chosen to balance several factors. Firstly, the spray solution and drying conditions should result in substantially homogeneous solid dispersions having the physical characteristics described above. Second, the spray solution and drying conditions should also allow the efficient manufacture of large quantities of such dispersions with large volumes of spray solution. The characteristics of the spray solution and drying conditions needed to achieve these goals are described in more detail below.

The spray solution determines the drug (e.g., DHM) loading of the resulting solid dispersion, and also affects whether the solid dispersion is homogeneous and the efficiency of production of the dispersions. The spray solution can contain the drug, a polymeric excipient, and a solvent.

The relative amounts of drug and polymer dissolved in the solvent are chosen to yield the desired drug to polymer ratio in the resulting solid dispersion, and the total dissolved solids content, e.g., the total solids concentration, of the spray solution can be sufficiently high, so that the spray solution results in efficient production of the solid amorphous dispersions. The total dissolved solids content refers to the amount of drug, polymer and other excipients dissolved in the solvent.

The solvent can be chosen to yield a substantially homogenous dispersion having a low residual solvent level. For example, the solvent can be chosen based on the following characteristics: (1) the active molecule(s) and excipients both are soluble, and may have high solubility in the solvent; (2) the solvent is volatile; and (3) the solution gels during solvent removal. The solubility of the drug in the solution can be great enough, so that the drug remains soluble at the solids content at which the solution gels.

In order to achieve dispersions that are substantially homogeneous, the solvent yields a spray solution in which the excipient(s) (e.g., polymer(s)) and active molecule(s) (drug, e.g., DHM) are both soluble and can be highly soluble. The drug (e.g., DHM) and polymer can be fully dissolved in the solvent in the spray solution prior to atomization, so that the polymer, drug, and solvent are intimately mixed at the molecular level. For example, the drug can have a solubility in the solvent at 25° C. of at least 0.5 wt %, at least 2.0 wt %, or at least 5.0 wt %. The polymer should be highly soluble (soluble to a large concentration) in the solvent as well. However, for polymers, this is best indicated by the nature of the solution it forms. A solvent can be chosen that solvates the polymer sufficiently for the polymer to not be highly aggregated and to form a visibly clear solution. High polymer aggregation is indicated by the solution being cloudy or turbid and by the solution scattering large amounts of light. Thus, the acceptability of a solvent can be determined by measuring the turbidity of the solution or the level of light scattering, as is known in the art. For example, for the polymer hydroxypropyl methyl cellulose acetate succinate (HPMCAS), acetone is a good solvent choice, forming a clear solution when the polymer is dissolved. In contrast, pure ethanol can be a poor choice for HPMCAS at a practical dissolved solids concentration, because only a small portion (about 20 to 30 wt %) of the HPMCAS is soluble in ethanol. This is seen in the resulting heterogeneous mixture that results when using ethanol as the solvent: a clear solution above an opaque solution of gelled, undissolved polymer. Good solvation can also lead to another property described below, gelation. If solvation is poor, the polymer precipitates, so that there is separation into a solvent-poor solid phase and a polymer-poor solution phase, rather than gelling, that is, remaining as a highly viscous liquid or a single-phase solid or semi-solid (polymer and solvent) material.

A solvent suitable for spray-drying can be any compound in which the drug(s) (e.g., DHM) and polymer(s) are mutually soluble. Examples of solvents include the following: alcohols, such as methanol (MeOH), ethanol (EtOH), n-propanol, iso-propanol, and butanol; ketones, such as acetone, methyl ethyl ketone (MEK), and methyl iso-butyl ketone; esters, such as ethyl acetate and propylacetate; and other solvents such as acetonitrile, methylene chloride, toluene, tetrahydrofuran (THF), cyclic ethers, and 1,1,1-trichloroethane. Lower volatility solvents, such as dimethyl acetamide and dimethylsulfoxide (DMSO), can also be used. Combinations or mixtures of solvents (e.g., multicomponent solvents), such as any of these exemplified solvents, e.g., 50% methanol and 50% acetone, can also be used, as can mixtures with water, as long as the polymer and drug are sufficiently soluble to make the spray-drying process practicable. In some cases it may be desired to add a small amount of water to aid solubility of the polymer in the spray solution.

DHM is soluble, for example, in acetone, tetrahydrofuran (THF), methanol, ethanol, and dimethylsulfoxide (DMSO), combinations of these, and one or more of these with a limited amount of water.

To achieve rapid solvent removal, and to keep the residual solvent level in the resulting solid amorphous dispersion low (preferably less than about 5 wt %), a volatile solvent can be chosen. For example, the boiling point of the solvent can be less than about 200° C., less than about 150° C., less than about 100° C., less than about 75° C., less than or equal to about 66° C., less than or equal to about 65° C., less than or equal to about 56° C., or less than about 50° C. However, if the solvent is too volatile, the solvent will evaporate too rapidly, resulting in particles that have low density unless the evaporation step is conducted at a low temperature. Operation at conditions where the temperature of the exhaust drying gas at the outlet is less than about 20° C. can be often impractical. In practice, acetone (56° C. boiling point), methanol (MeOH) (65° C. boiling point), and tetrahydrofuran (THF) (66° C. boiling point) work well for a variety of drugs and active ingredients, such as DHM.

The solvent is chosen to cause the atomized droplets of drug (e.g., DHM), polymer, and solvent to gel prior to solidification during the evaporation process. Initially, the spray solution is a homogeneous solution of dissolved drug and polymer in the solvent. When the spray solution is sprayed into the drying chamber, the spray solution is atomized into liquid droplets. The solvent begins to rapidly evaporate from the liquid droplets, causing the concentration of the dissolved drug and polymer to increase in the droplet. As the solvent continues to evaporate, there are three possible scenarios: (1) the polymer concentration in the droplet exceeds the gel point of the polymer so as to form a homogeneous gel; (2) the concentration of the dissolved drug in the droplet exceeds the solubility of the drug in the solution in the droplet, causing the drug to phase separate from the solution; or (3) the concentration of the polymer in the droplet exceeds the solubility of the polymer in the solution in the droplet, causing the polymer to phase separate from the solution. Homogeneous solid dispersions are most readily formed when the solvent and concentrations of polymer and drug are chosen such that, as solvent is evaporated, the polymer, drug, and solvent form a homogeneous gel prior to the drug phase separating or the polymer precipitating, i.e., scenario (1) above. In contrast, if the drug or polymer phase separate prior to the polymer gelling, then it becomes more difficult to choose spray drying conditions which will yield a substantially homogeneous dispersion. Gelation of the solution prior to reaching the solubility limit of the drug greatly slows the drug phase separation process, providing adequate time for solidification of the particles in the spray drying process without significant phase separation.

By choosing a solvent which causes the polymer to gel, the concentration of the polymer will exceed the gel point of the polymer as the solvent evaporates from the solvent, resulting in a homogeneous gel of the drug (e.g., DHM), polymer, and solvent. When this occurs, the viscosity of the solution in the droplet increases rapidly, immobilizing the drug and polymer in the droplet notwithstanding the presence of the solvent. As additional solvent is removed, the drug and polymer remain homogeneously distributed throughout the droplet, resulting in a substantially homogeneous solid dispersion.

Alternatively, the solvent and the polymer and drug (e.g., DHM) concentrations may be chosen such that, as the solvent evaporates, the drug concentration exceeds the drug solubility in the solvent—that is, the drug supersaturates. For such a case, the drug may have a relatively low solubility in the solvent, but the polymer may have a high solubility and gel at the time of saturation of the drug. Such a system may yield a satisfactory solid amorphous dispersion (i.e., with the drug not having phase separated as an amorphous or crystalline phase), so long as the time during which the solution has a drug concentration above the point where it will ultimately phase separate from the solution (i.e., the drug is supersaturated) and the solution is still liquid (i.e., gelation has not yet occurred) is sufficiently short, so that the drug does not substantially phase separate prior to gelation.

Alternatively, the drug (e.g., DHM) may precipitate and the polymer gel at the same time, so that the drug and the polymer remain homogeneously distributed throughout the droplet, resulting in a substantially homogeneous solid dispersion.

For example, the size of 50 wt %, 80 wt %, 90 wt %, or more of the particles in a spray-dried dispersion (SDD) powder including DHM can be within the range of from 0.1 to 100 μm, from 0.5 to 50 μm, from 1 to 25 μm, or from 10 to 15 μm, or can be about 10 μm, or can be at least 1 μm, at least 5 μm, or at least 10 μm. A larger particle size can facilitate the handling and processing of a powder. For example, the particle size distribution of the particles in an SDD powder can have a polydispersity index (PDI) of 4 or less, 2 or less, 1.5 or less, 1.2 or less, 1.1 or less, or 1.05 or less.

Greater concentration of DHM and polymer in the solution to be sprayed can result in larger particles. Larger scale spray dryers can have atomization nozzles, for example, atomization nozzles with larger orifices, that can produce larger liquid droplets, leading to larger resulting particle size. Commercially supplied pure DHM can be entirely (100%) or nearly entirely crystalline.

The crystallinity of the DHM in the spray-dried dispersion powder can be qualitatively assessed or quantitatively measured by techniques such as polarized light microscopy (PLM), differential scanning calorimetry (DCS), and powder X-ray diffraction (P-XRD or PXRD). The DHM in the spray-dried dispersion powder can have a crystallinity of less than or equal to 90%, 80%, 60%, 50%, 40%, 30%, 20%, 25%, 20%, 15%, 10%, 7%, 5%, 3%, 2%, or 1%. The DHM in the spray-dried dispersion powder can be amorphous.

### Mixing

It is important that the spray solution is prepared, so as to achieve a homogeneous spray solution in which all of the drug (e.g., DHM) and matrix material, e.g., polymer, are completely dissolved. In general, the drug and polymer are added to the solvent and mechanically mixed or agitated over a period of time. Exemplary mixing processes include submerged impellers or agitators. The solution is mixed for a period of time, such as from 0.1 to 10 hours, 0.5 to 8 hours, or 4 to 8 hours, to ensure that all of the polymer and drug have dissolved. In an embodiment, the drug and polymer are mixed with the solvent using a separate mixing device, such as a high shear powder disperser, jet mixer, or line blender.

### Process Conditions

The manner in which the solvent is evaporated from the spray solution also affects the density and size of the solid amorphous dispersion particles, as well as whether the solid amorphous dispersion is homogeneous. A difficulty in removing the solvent is that factors which tend to favor formation of homogeneous particles may lead to particles having an undesirably low density, and vice versa. To form a homogeneous dispersion, it is desired to remove solvent rapidly. Since the spray solution is a homogeneous mixture of drug (e.g., DHM), polymer and solvent, the solvent should be removed on a time frame that is short relative to the time required for the drug and polymer to separate from each other. On the other hand, to form dense particles, solvent should be removed slowly. However, this may yield particles that are non-homogeneous and/or have undesirably high residual solvent levels.

The solvent can evaporate sufficiently rapidly, such that the droplets are essentially solid when they reach the outlet of the drying chamber and have a residual solvent content of less than 10 wt %. The large surface-to volume ratio of the droplets and the large driving force for the evaporation of solvent can lead to actual drying times of a few seconds or less, for example, less than 0.1 second. Drying times to a residual solvent level of less than 1, 2, 5, or 10 wt % can be less than 100 seconds, less than 20 seconds, less than 1 second, or less than 0.1 second.

The drying gas input to the spray drying chamber may be virtually any gas, but to minimize the risk of fire or explosion, because of ignition of flammable vapors, and to minimize undesirable oxidation of the drug (e.g., DHM), (concentration-enhancing) polymer, or other materials in the dispersion, an inert gas such as nitrogen, nitrogen-enriched air, or argon can be used. In addition, the drying gas entering the drying chamber at the inlet may contain small amounts of the solvent in vapor form.

The temperature of the spray solution may be selected based on the solubility characteristics and stability of the constituents of the spray solution. In general, the spray solution can be held at a temperature ranging from about 0° C. to 50° C., and can be maintained near room temperature. The temperature may be raised to improve the solubility of the drug (e.g., DHM) or polymer in the solution. In addition, the temperature of the spray solution may be set at an elevated temperature to provide additional heat to the drying process and to further increase the rate of evaporation of solvent from the droplets. The temperature may be lowered, if needed to improve the stability of the drug in the spray solution.

The feed rate of the spray solution into the spray drying chamber will depend on a variety of factors, such as the drying gas inlet temperature (T_{IN}), drying gas flow rate, the size of the drying chamber, and the size of (e.g., the internal diameter of the passage or orifice through) the atomizer. In the laboratory, the feed rate of the spray solution can be, for example, from 0.05 or 0.3 to 1 kg/hr. The feed rate of the spray solution when spray drying using a Niro PSD-2 Spray dryer may range from 10 to 85 kg/hr or from 50 to 75 kg/hr. The spray drying process according to the invention can have particular utility as the feed rate of the spray solution increases, allowing production of increasing quantities of product. In an embodiment, the feed rate of the spray solution is at least 50 kg/hr, at least 100 kg/hr, at least 200 kg/hr, or at least 400 kg/hr. In an embodiment, the spray solution feed rate may range from 400 kg/hr to 600 kg/hr. The feed rate of the spray solution can be controlled, in conjunction with T_{IN}, so as to achieve efficient spray drying, high product yield, and good particle characteristics.

In an embodiment, warm nitrogen (N₂) gas is introduced with the spray solution that includes DHM and polymer that passes through the atomizer into the drying chamber. The drying chamber can be maintained at a temperature that is at least 20° C. greater than the boiling point of the solvent. For example, acetone has a boiling point (at standard atmospheric pressure) of 56° C., so that the drying chamber can be maintained at a temperature of 76° C. or greater. For example, tetrahydrofuran (THF) has a boiling point (at standard atmospheric pressure) of 66° C., so that the drying chamber can be maintained at a temperature of 86° C. or greater.

The spray dried dispersion processing parameters can include the inlet temperature, outlet temperature, drying gas temperature, temperature of the drying chamber, drying gas flow rate, inlet feed rate, chamber diameter, atomizer size (e.g., atomizer diameter), pressure drop across the atomizer, and spray drying chamber (drying chamber) pressure, which can be optimized.

Additional process parameters are the drug (e.g., DHM) loading and the total solids concentration in the spray solution, i.e., the solution to be forced through the atomizer into the drying chamber. The DHM loading is the ratio of the mass of DHM to the mass of DHM, polymer, and other solid materials in the spray solution. For example, the DHM loading can be in the range of from 10% to 50%, from 20% to 40%, or about 30%. The total solids concentration is the ratio of the mass of all solids (e.g., DHM, polymer, and other solid material) to the mass of those solids plus the mass of solvent in the spray solution. For example, the total solids concentration can be from 5% to 15%, or about 10%.

Advantages of the Spray Drying process

In the present invention, the use of spray dried dispersion technology (SDD) in the preparation of dosage forms to more effectively deliver flavonoids, e.g., DHM, has several advantages. Spray drying is an industrial technology that is scalable. It allows for DHM to be formulated with a wide variety of excipients and matrix materials to allow for maximum bioavailability and efficacy for the desired administration route. The spray drying process can trap active molecules (e.g., DHM) in a high-energy amorphous state as needed to significantly improve bioavailability and sustained concentrations in the bloodstream as needed.

### Administration

The resulting formulations of embodiments of the present invention are useful and suitable for delivery in animals and humans and may be administered by a variety of methods. Such methods include, by way of example and without limitation, oral, nasal, buccal, rectal, ophthalmic, otic, urethral, vaginal, or sublingual dosage administration. Such methods of administration and others contemplated within the scope of the present invention are known to the skilled artisan. In vivo stability of the present formulation may vary according to the physiological environment to which it is exposed and the excipients and matrix material used. Therefore, the necessity for or frequency of readministration may be different for various formulations.

The formulations of embodiments of the present invention may be provided in a variety of ways, for example, powder, tablet, and capsule dosage forms. Additional components that would not significantly prohibit the spray drying process may be added to the spray solution prior to spray drying. That is, such additional components should still allow for formulation using the spray drying process.

For oral, buccal, and sublingual administration, the formulation may be in the form of a gel cap, caplet, tablet, capsule, suspension, or powder. Alternatively, the formulation may be in the form of a mixture with or suspension in, e.g., a DHM-containing powder according to an embodiment of the invention mixed with or suspended in, a consumable (edible) liquid (e.g., an aqueous liquid, such as water), such as a drink or liquid concentrate. Alternatively, the formulation may be in the form of a mixture with or suspension in, e.g., a DHM-containing powder according to an embodiment of the invention mixed with or suspended in, an edible gel. For rectal administration, the formulation may be in the form of a suppository, ointment, enema, tablet, or cream for release of compound into the intestines, sigmoid flexure, and/or rectum.

In solid dosage forms, the compounds can be combined with one or more carriers, for example, one or more of the following: binders, such as acacia, corn starch, or gelatin; disintegrating agents, such as corn starch, guar gum, potato starch, or alginic acid; lubricants, such as stearic acid or magnesium stearate; and inert fillers, such as lactose, sucrose, or corn starch.

It is contemplated that either one or a combination of long-acting, sustained release, controlled release, or slow release dosage forms may be used in the present invention. The course and duration of administration of and the dosage requirements for the formulation of the present invention will vary according to the subject being treated, the formulation used, the method of administration used, the severity of the condition being treated, the co-administration of other drugs, and other factors.

### Dissolution/Kinetics Studies

In an embodiment, the DHM in the spray-dried dispersion powder does not dissolve in and/or is not solubilized by an aqueous solution having a pH of at most (i.e., less than or equal to) 4.8, 4.5, 4, 3.5, 3.2, 3, 2.7, 2.5, 2.3, 2, 1.8, 1.5, or 1. The chyme that is expelled by the stomach, through the pyloric valve, has a pH of approximately 2. Gastric juices lead to material in the stomach having a pH in the range of from 1.5 to 3.5, and this low pH in the stomach and the enzymes active in the stomach at this low pH may result in degradation of DHM and quenching of DHM activity.

In an embodiment, the DHM in the spray-dried dispersion powder dissolves in and/or is solubilized by water (pH of 7) and/or an aqueous solution having a pH of at least (i.e., greater than or equal to) 5, 5.3, 5.5, 5.8, 6, 6.2, 6.5, 6.7, 7, 7.2, or 7.5. Bile released into the duodenum and/or pancreatic secretions of sodium bicarbonate increase the pH of the chyme. For example, the pH of chyme and material in the intestine (bowel) can range from 5.5 to 7, for example, can be 7. The dissolution and/or solubilization of the DHM in the spray-dried dispersion powder in the intestine, for example, the small intestine, can result in the DHM being absorbed by the wall of the intestine, for example, the wall of the small intestine, and into the blood.

For example, hydroxypropyl methyl cellulose acetate succinate (HPMCAS) is insoluble in an aqueous solution of acidic (low) pH, but is soluble in an aqueous solution of neutral or alkaline (high) pH. Therefore, a spray-dried dispersion powder including HPMCAS and DHM can reduce release of, delay release of, or retain the DHM at an acidic (low) pH, e.g., a pH of 3.5 or less, but release the DHM at a neutral or alkaline (high) pH, e.g., a pH of 7 or greater.

A pH buffering agent can be included in such spray-dried dispersion (SDD) powder. Inclusion of an acidic component in such a spray-dried dispersion (SDD) powder, such as an acidic pH buffering agent (i.e., a buffering agent that maintains an acidic pH, a pH of less than 7), e.g., citric acid or a citrate salt (e.g., a sodium citrate, a potassium citrate, calcium citrate, and/or combinations), can stabilize an aqueous solution formed with the spray-dried dispersion powder, so that the DHM is not released into the aqueous solution or so that the release of the DHM into the aqueous solution is delayed.

The polymer matrix material can be selected, so that it is soluble in the solvent that is used to form the spray solution that is forced through the atomizer in the spray drying process, and so that it is moderately soluble (e.g., from 0.01 g/100 mL to 3 g/100 mL, or from 0.1 g/100 mL to 1 g/100 mL) in water. Moderate solubility in water allows the polymer matrix material to dissolve in the body of an organism and release the DHM.

The dissolution and release kinetics of DHM can be studied under different conditions; three protocols are described as follows.[22]

Release Kinetics in Vitro: Simulated gastric fluid (FaSSGF) and intestinal fluids (FaSSIF and FeSSIF) are prepared according to the manufacturer's instructions. Dissolution tests are performed with spray dried DHM-containing powders or tablets with the appropriate controls.

Release under Gastric Conditions: DHM-containing spray dried powder samples are suspended in prewarmed FaSSGF (37° C.) to achieve a drug (DHM) concentration of roughly 10-100× the previously determined equilibrium solubility in the FaSSGF fluid (e.g., 75 μg/mL) by pipetting up and down vigorously multiple times. The samples are incubated for the duration of the study (e.g., 30 min) at 37° C. (NesLab RTE-111 bath circulator, Thermo Fisher Scientific, Waltham, Mass.) without agitation to mimic physiological gastric conditions and transition time in the stomach. Aliquots can be taken, for example, at 1, 5, 10, 15, 30, 60, 120, and 360 min. To analyze the free DHM concentration, each aliquot can be centrifuged at 28000 g for 5 min to pellet suspended particles. The supernatant is frozen and lyophilized; the remaining solids are reconstituted in, for example, 2:8 THF (tetrahydrofuran):acetonitrile to dissolve DHM and precipitate out lipids and salts from the release media. The samples are then diluted as appropriate to fall within the detection range and analyzed by high-performance liquid chromatography (HPLC), with the mobile phase as 80:20 H₂O:acetonitrile (each with 0.05% trifluoroacetic acid), and with detection with UV-Vis at 290 nm. The concentration of DHM is then calculated based on a calibration curve.

Release under Intestinal Conditions: DHM-containing spray dried powder samples are suspended in prewarmed (37° C.) Fed State Simulated Intestinal Fluid (FeSSIF) or Fasted State Simulated Intestinal Fluid (FaSSIF) to achieve a drug (DHM) concentration of roughly 10-100× the previously determined equilibrium solubility in the FeSSIF or FaSSIF fluid by pipetting up and down vigorously multiple times. The equilibrium solubility of crystalline DHM in FeSSIF was measured to be about 140 μg/mL, and the equilibrium solubility of crystalline DHM in FaSSIF is about 50 μg/mL. Aliquots are taken at, for example, 1, 5, 10, 15, 30, 60, 120, and 360 min and centrifuged at, for example, 28000 g for 10 min. The supernatant is frozen and lyophilized; the remaining solids are reconstituted in, for example, 2:8 THF (tetrahydrofuran):acetonitrile to dissolve DHM and precipitate out lipids and salts from the release media. The samples are then diluted as appropriate to fall within the detection range and analyzed by HPLC, with the mobile phase as 80:20 H₂O:acetonitrile (each with 0.05% trifluoroacetic acid), and with detection with UV-Vis at 290 nm. The concentration of DHM is then calculated based on a calibration curve.

FaSSIF is a biorelevant intestinal media representing the fasted state intestinal fluid, and FeSSIF is another biorelevant intestinal media representing the fed state intestine fluid. FaSSIF and FeSSIF have different compositions. For example, components of FaSSIF include 3 mM taurocholate, 0.75 mM phospholipids, 148 mM sodium, 106 mM chloride, and 29 mM phosphate, while components of FeSSIF include 15 mM taurocholate, 3.75 mM phospholipids, 319 mM sodium, 203 mM chloride, and 144 mM acetic acid. In in vivo tests, the presence of food changes the pH and composition of fats and surfactants in the intestinal fluid. FaSSIF has a higher pH (6.5) than FeSSIF (5.0) and has lower levels of fat.

The intestine can be the site of absorption for oral dosage forms, thus understanding the solubility of a drug or active ingredient in the intestinal fluid can be important.

For example, the dissolution kinetics of DHM in a spray-dried dispersion formulation in an embodiment of the present invention in in vitro dissolution tests in simulated fasted state fluid can be increased by 5%, 10%, 15%, 20%, 30%, 40%, 50%, 60%, 100%, or 250% after 15 minutes over that of pure DHM.

For example, the dissolution kinetics of DHM in a spray-dried dispersion formulation in an embodiment of the present invention in in vitro dissolution tests in simulated fed state fluid can be increased by 5%, 10%, 15%, 20%, 30%, 40%, 50%, 60%, 100%, or 250% after 15 minutes over that of pure DHM.

For example, the dissolution kinetics of DHM in a spray-dried dispersion formulation in an embodiment of the present invention in in vitro dissolution tests in simulated fasted state fluid can be increased by 5%, 10%, 15%, 20%, 30%, 40%, 50%, 60%, 100%, or 250% after 30 minutes over that of pure DHM.

For example, the dissolution kinetics of DHM in a spray-dried dispersion formulation in an embodiment of the present invention in in vitro dissolution tests in simulated fed state fluid can be increased by 5%, 10%, 15%, 20%, 30%, 40%, 50%, 60%, 100%, or 250% after 30 minutes over that of pure DHM.

For example, the dissolution kinetics of DHM in a spray-dried dispersion formulation in an embodiment of the present invention in in vitro dissolution tests in simulated fasted state fluid can be increased by 5%, 10%, 15%, 20%, 30%, 40%, 50%, 60%, 100%, or 250% after 60 minutes over that of pure DHM.

For example, the dissolution kinetics of DHM in a spray-dried dispersion formulation in an embodiment of the present invention in in vitro dissolution tests in simulated fed state fluid can be increased by 5%, 10%, 15%, 20%, 30%, 40%, 50%, 60%, 100%, or 250% after 60 minutes over that of pure DHM.

For example, the dissolution kinetics of DHM in a spray-dried dispersion formulation in an embodiment of the present invention in in vitro dissolution tests in simulated fasted state fluid can be increased by 5%, 10%, 15%, 20%, 30%, 40%, 50%, 60%, 100%, or 250% after 120 minutes over that of pure DHM.

For example, the dissolution kinetics of DHM in a spray-dried dispersion formulation in an embodiment of the present invention in in vitro dissolution tests in simulated fed state fluid can be increased by 5%, 10%, 15%, 20%, 30%, 40%, 50%, 60%, 100%, or 250% after 120 minutes over that of pure DHM.

For example, the dissolution kinetics of DHM in a spray-dried dispersion formulation in an embodiment of the present invention in in vitro dissolution tests in simulated fasted state fluid can be increased by 5%, 10%, 15%, 20%, 30%, 40%, 50%, 60%, 100%, or 250% after 360 minutes over that of pure DHM.

For example, the dissolution kinetics of DHM in a spray-dried dispersion formulation in an embodiment of the present invention in in vitro dissolution tests in simulated fed state fluid can be increased by 5%, 10%, 15%, 20%, 30%, 40%, 50%, 60%, 100%, or 250% after 360 minutes over that of pure DHM.

### Animal PK Studies

DHM-containing samples (e.g., spray-dried dispersions in an embodiment of the present invention) can be administered (e.g., through oral gavage) to an animal (e.g., a rat or a mouse) at 10 mg DHM/kg body weight, 75 mg DHM/kg body weight, or another dosage in an in vivo study, and a pharmacokinetic study can be carried out to evaluate animal pharmacokinetics. The plasma concentration of DHM can be determined, for example, using a Waters Acquity ultra performance liquid chromatography system equipped with an electrospray ionization mass spectrometry system (Waters, Milford, Mass.), in accordance with a previous report [23], or an equivalent analytical analysis system.

An animal dosed with a spray-dried dispersion powder containing DHM according to the present invention can show increased blood maximum concentrations, relative to dosing with pure DHM powder, of 5%, 10%, 15%, 20%, 30%, 40%, 50%, 60%, 100%, or 250%. The area under the curve (AUC) for 24 hours can be increased by 5%, 10%, 15%, 20%, 30%, 40%, 50%, 60%, 100%, or 250% over the value associated with dosing with pure DHM powder.

### EXAMPLES

The following examples provide a detailed description of embodiments of the invention. It is recognized that departures from the disclosed embodiments may be made within the scope of the invention and that obvious modifications will occur to a person skilled in the art. The claims and specification should not be construed to narrow the full scope of protection to which the invention is entitled.

### Example 1

### Preparation and Characterization of a DHM-HPMCAS Spray Dried Formulation

An amount of dihydromyricetin (DHM) sufficient to provide an effective amount of the formulation was mixed with a known amount of hydroxypropylmethylcellulose acetate succinate (HPMCAS) polymer (Dow AFFINISOL 126), and this mixture was dissolved in acetone as the solvent at a total solids concentration of 10% (w/w). A Büchi B-290 Spray Dryer with a B-295 inert loop was used. The vessel containing the solution was then connected via tubing to a spray drying nozzle assembly (atomizer) which is placed within a spray drying chamber. The solution was then pumped through the spray drying nozzle assembly into the chamber along with hot drying gases such as nitrogen and air, which could be at a temperature between 25° C. and 140° C. The feed rate was 7.5 mL/min (450 mL/hr); the inert gas pressure was 70 psi; the inert gas flow rate was ˜500 L/hour; the inlet temperature was 90° C.; the outlet temperature was ˜55° C.; the inert loop condenser temperature was −15° C.; and the aspirator rate was 90% (which was approximately equivalent to a gas circulation volumetric rate of 35 m³/hr).

The spray dried dispersion (SDD) powder was then collected in a vial located at the bottom of the cyclone separator following the drying chamber. The SDD powder can be further dried in a secondary step and/or processed into tablets, capsules, or another dosage form.

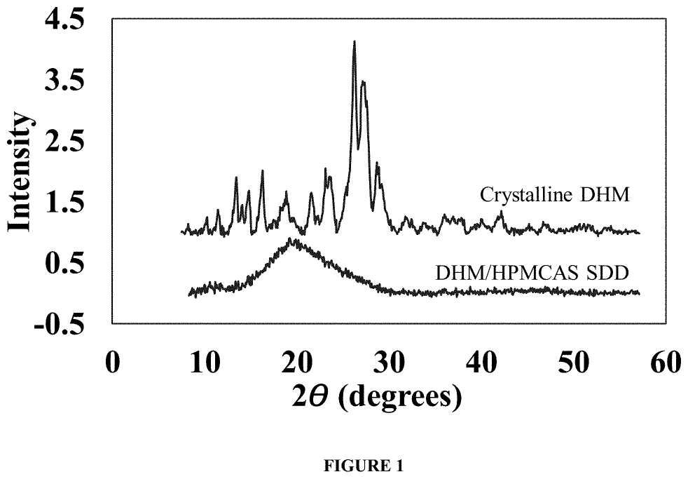

The result of a Powder X-Ray Diffraction (PXRD) measurement is presented in FIG. 1, which shows PXRD traces for both the starting crystalline DHM material and the SDD formulation. In FIG. 1, the crystalline DHM trace is shifted up by 1 intensity unit, so that it does not overlap the SDD trace. PXRD detects crystallinity in a powder sample by measuring the diffraction of X-rays by a sample's crystalline lattice in accordance with Bragg's law. Here, it was observed that the starting DHM material had a well-defined crystalline lattice, as evidenced by the sharp peaks in its PXRD trace. In contrast, the exemplary SDD material exhibited only a single broad trace and lacked the signature peaks of the starting crystalline DHM material. This indicated amorphous DHM in the SDD formulation.

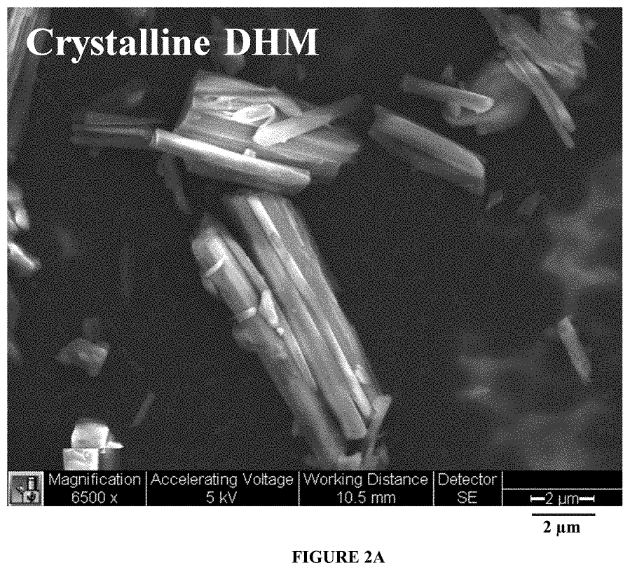

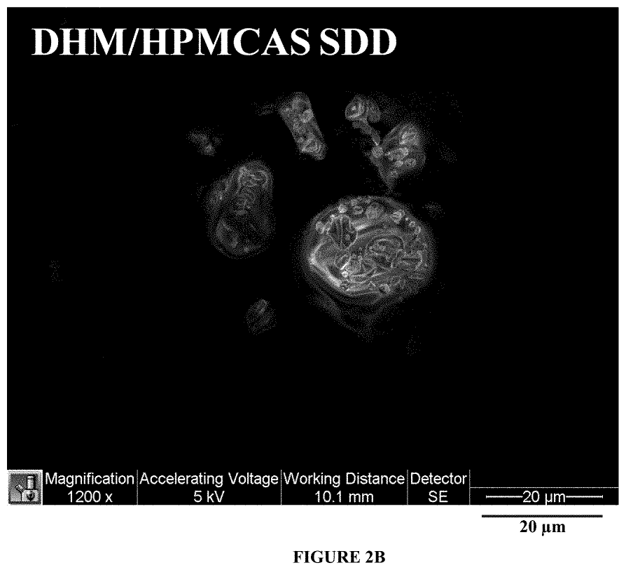

Scanning Electron Microscopy (SEM) micrographs of the starting crystalline DHM and the SDD formulation are presented in FIGS. 2A and 2B, respectively. A rod morphology of the DHM crystals was observed in the starting material (FIG. 2A), whereas the SDD material exhibited the morphology of a collapsed droplet (FIG. 2B), which was produced by the rapid evaporation of solvent during the spray drying process. Scale bars in FIGS. 2A and 2B indicate the size of features in the micrographs. The SDD material did not show evidence of DHM crystals; instead, FIG. 2B indicated that a uniform dispersion of amorphous DHM and the HPMCAS polymer was formed in the SDD material.

In the exemplary spray-dried dispersion (SDD) powder produced, the drug (DHM) loading in the particles was 20%, i.e., the weight ratio of DHM:polymer was 20:80. (For example, in other cases, the weight ratio of DHM to polymer may be about 5:95, 10:90, 30:70, 40:60, 50:50, 60:40, or 80:20.) The crystallinity of the DHM in the spray-dried dispersion powder was at most about 5% or 10%, based on the detection limit of the Powder X-Ray Diffraction (PXRD) measurement. The exemplary SDD powder was confirmed to be X-ray amorphous by the PXRD measurement (FIG. 1), as well as by the Scanning Electron Microscopy (SEM) image (FIG. 2B).

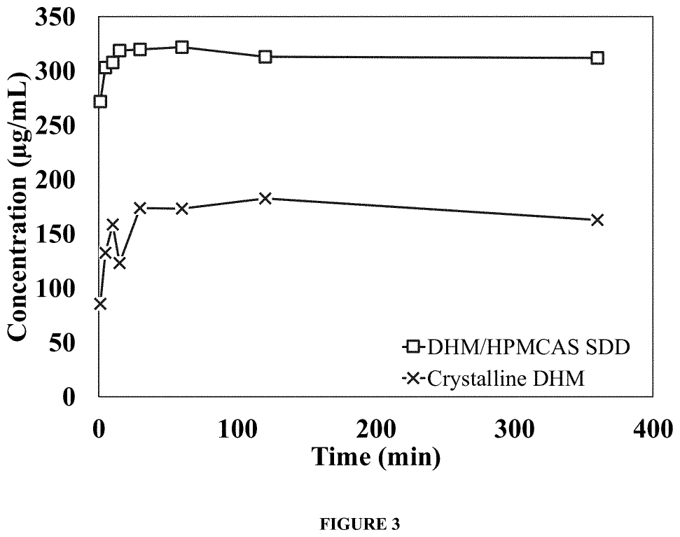

The result of in vitro release data is shown in FIG. 3, which compares the starting crystalline DHM material with the SDD formulation over a 6 hour time period with the following sampling timepoints: 1, 5, 10, 15, 30, 60, 120, and 360 minutes. The release study was performed in fed state simulated intestinal fluid (FeSSIF, Biorelevant.com Ltd.) at 37° C. For each study shown by the corresponding data points in FIG. 3, the starting crystalline DHM material or the SDD formulation was loaded into the prepared FeSSIF fluid at a DHM concentration of 1400 μg/mL, 10 times the previously measured equilibrium solubility of crystalline DHM in FeSSIF (140 μg/mL). (The excess DHM loaded into the release media enables detection and quantitation of potentially supersaturated dissolved DHM concentrations that a given formulation may provide.) At the completion of the release studies (360 minute timepoint), it was observed that the SDD formulation produced a supersaturated DHM concentration which was roughly double that of the equilibrium concentration of crystalline DHM (see FIG. 3). By contrast, the crystalline DHM approximately reached its previously determined equilibrium concentration (see FIG. 3).

These release studies showed that the SDD formulation will release a concentration of dissolved DHM into a patient's intestine that is much greater (approximately double) the concentration of dissolved DHM released by crystalline DHM. The greater concentration of dissolved DHM released into the patient's intestine results in the SDD formulation promoting uptake of DHM into the overall system (body) of the patient that is greater than the DHM uptake when crystalline DHM is administered, by way of an increased concentration driving force of the DHM across the membrane of the intestine.

### Example 2

### Preparation and Characterization of a DHM-HPMCAS Spray Dried Formulation

Similar to Example 1, another spray-dried dispersion (SDD) formulation was prepared as follows.

An amount of dihydromyricetin (DHM) sufficient to provide an effective amount of the formulation was mixed with a known amount of hydroxypropylmethylcellulose acetate succinate (HPMCAS) polymer (Dow AFFINISOL 126), and this mixture was dissolved in acetone as the solvent at a total solids concentration of 10% (w/w). The weight ratio of DHM to polymer was 40:60. A Büchi B-290 Spray Dryer with a B-295 inert loop was used. The vessel containing the solution was then connected via tubing to a spray drying nozzle assembly (atomizer) which is placed within a spray drying chamber. The solution was then pumped through the spray drying nozzle assembly into the chamber along with hot drying gases such as nitrogen and air, which could be at a temperature between 25° C. and 140° C. The feed rate was 7.5 mL/min (450 mL/hr); the inert gas pressure was 70 psi; the inert gas flow rate was ˜500 L/hour; the inlet temperature was 90° C.; the outlet temperature was ˜55° C.; the inert loop condenser temperature was −15° C.; and the aspirator rate was 90% (which was approximately equivalent to a gas circulation volumetric rate of 35 m³/hr).

The spray dried dispersion (SDD) powder was then collected in a vial located at the bottom of the cyclone separator following the drying chamber. The SDD powder can be further dried in a secondary step and/or processed into tablets, capsules, or another dosage form.

Thus, in the exemplary spray-dried dispersion (SDD) powder produced, the drug (DHM) loading in the particles was 40%, i.e., the weight ratio of DHM:polymer was 40:60.

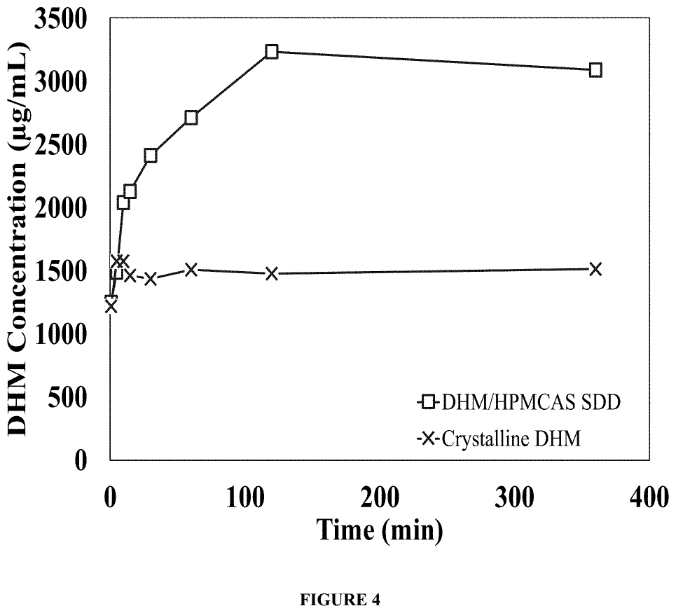

The result of in vitro release data is shown in FIG. 4, which compares the starting crystalline DHM material with the SDD formulation over a 6 hour time period with the following sampling timepoints: 1, 5, 10, 15, 30, 60, 120, and 360 minutes. The release study was performed in fed state simulated intestinal fluid (FeSSIF, Biorelevant.com Ltd.), to which 1% v/v Tween 20 (Polysorbate 20) was added. The fluid was maintained at a temperature of 37° C. for the duration of the experiment. For each study shown by the corresponding data points in FIG. 4, the starting crystalline DHM material or the SDD formulation was loaded into a separate volume of prepared FeSSIF fluid at a concentration of roughly 7 mg/mL, 50 times the previously measured equilibrium solubility of DHM in FeSSIF (140 μg/mL). (The excess DHM loaded into the release media enables detection and quantitation of potentially supersaturated dissolved DHM concentrations that a given formulation may provide.) At the completion of the release studies (360 minute timepoint), it was observed that the SDD formulation produced a supersaturated DHM concentration which was roughly double that of the crystalline DHM (see FIG. 4). The solubility of DHM was increased by the presence of the Tween 20 (Polysorbate 20) surfactant. However, the SDD formulation still produced a concentration of DHM that was about double that of the crystalline DHM.

These release studies further established that the SDD formulation will release a concentration of dissolved DHM into a patient's intestine that is much greater (approximately double) the concentration of dissolved DHM released by crystalline DHM. The greater concentration of dissolved DHM released into the patient's intestine results in the SDD formulation promoting uptake of DHM into the overall system (body) of the patient that is greater than the DHM uptake when crystalline DHM is administered, by way of an increased concentration driving force of the DHM across the membrane of the intestine.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### BIBLIOGRAPHY

1. Powell, B. A. R., COMPOSITIONS AND METHODS FOR PREVENTING AND RECOVERY FROM DETRIMENTAL EFFECTS OF ALCOHOL CONSUMPTION, U.S. Pat. No. 9,603,830, (Mar. 28, 2017), THRIVEPLUS LLC: USA.
2. Shen, Y., et al., Dihydromyricetin As a Novel Anti Alcohol Intoxication Medication. The Journal of Neuroscience, 2012. 32(1): p. 390-401.
3. [symbol] and [symbol] , Application of dihydromyricetin in preparation of food, cosmetics or medicine in Google Patents. 2007, [symbol] : China.
4. [symbol] , Dihydromyricetin cyclodextrin inclusion compound and preparation method thereof in Google Patents. 2015, [symbol] : China.
5. [symbol] and [symbol] , Method for preparing dihydromyricetin solid lipid granules in Google Patents. 2016, [symbol] : China.
6. Yao, M.-j. and J.-h. Huang, Study on the microencapsulation of dihydromyricetin. Journal of Jishou University (Natural Science Edition), 2007. 28(3).
7. Davies, D. L., et al., Recent advances in the discovery and preclinical testing of novel compounds for the prevention and/or treatment of alcohol use disorders. Alcoholism: Clinical and Experimental Research, 2013. 37(1): p. 8-15.
8. Liang, J., et al., Dihydromyricetin prevents fetal alcohol exposure-induced behavioral and physiological deficits: the roles of GABAA receptors in adolescence. Neurochemical research, 2014. 39(6): p. 1147-1161.
9. Shen, Y., et al., Dihydromyricetin as a novel anti-alcohol intoxication medication. Journal of Neuroscience, 2012. 32(1): p. 390-401.
10. Ji, Y., J. Li, and P. Yang, Effects of fruits of Hovenia dulcis Thunb on acute alcohol toxicity in mice. Zhong yao cai=Zhongyaocai=Journal of Chinese medicinal materials, 2001. 24(2): p. 126-128.
11. Fang, H.-L., et al., Treatment of chronic liver injuries in mice by oral administration of ethanolic extract of the fruit of Hovenia dulcis. The American journal of Chinese medicine, 2007. 35(04): p. 693-703.
12. Hase, K., et al., Hepatoprotective Effect of Hovenia dulcis THUNB. on Experimental Liver Injuries Induced by Carbon Tetrachloride or D-Galactosamine: Lipopolysaccharide. Biological and pharmaceutical Bulletin, 1997. 20(4): p. 381-385.
13. Ji, Y., et al., Effects of Hovenia dulcis Thunb on blood sugar and hepatic glycogen in diabetic mice. Zhong yao cai=Zhongyaocai=Journal of Chinese medicinal materials, 2002. 25(3): p. 190-191.
14. Okuma, Y., et al., Effect of extracts from Hovenia dulcis Thunb. alcohol concentration in rats and men administered alcohol. Journal of Japanese Society of Nutrition and Food Science (Japan), 1995.
15. WANG, X.-y. and Z.-t. JIANG, RESEARCH PROGRESS IN NATURAL ANTIOXIDANT DIHYDROMYRICETIN[J]. Food Research and Development, 2007. 2: p. 056.
16. Zhang, X., et al., Scavenging effect of dihydromyricetin on the free radicals by ESR. Modern Food Science and Technology, 2010. 26(10): p. 1040-1042, 1070.
17. Liu, B., et al., Characterization and antioxidant activity of dihydromyricetin-lecithin complex. European Food Research and Technology, 2009. 230(2): p. 325-331.
18. Aungst, B. J., Absorption enhancers: applications and advances. The AAPS journal, 2012. 14(1): p. 10-18.
19. Thanou, M., J. Verhoef, and H. Junginger, Oral drug absorption enhancement by chitosan and its derivatives. Advanced drug delivery reviews, 2001. 52(2): p. 117-126.
20. Whitehead, K., N. Karr, and S. Mitragotri, Safe and effective permeation enhancers for oral drug delivery. Pharmaceutical research, 2008. 25(8): p. 1782-1788.
21. Whitehead, K. and S. Mitragotri, Mechanistic analysis of chemical permeation enhancers for oral drug delivery. Pharmaceutical research, 2008. 25(6): p. 1412-1419.
22. Zhang, Y., et al., Design and Solidification of Fast-Releasing Clofazimine Nanoparticles for Treatment of Cryptosporidiosis. Molecular pharmaceutics, 2017. 14(10): p. 3480-3488.
23. Onoue, S., et al., Self-micellizing solid dispersion of cyclosporine A with improved dissolution and oral bioavailability. Eur J Pharm Sci, 2014. 62: p. 16-22.

1. A dihydromyricetin (DHM) dosage powder, comprising:
dihydromyricetin (DHM) and
a matrix material.

2. The DHM dosage powder of claim 1, wherein the matrix material is a polymeric matrix material.

3. The DHM dosage powder of any one of claims 1 through 2, wherein the matrix material comprises a linear polymer.

4. The DHM dosage powder of any one of claims 1 through 3, wherein the matrix material does not comprise a cyclic polymer.

5. The DHM dosage powder of any one of claims 1 through 4, wherein the matrix material comprises a polymer of molecular weight of at least 5 kDa, 10 kDa, or 20 kDa.

6. The DHM dosage powder of any one of claims 1 through 5, wherein the matrix material comprises cellulose or a cellulose derivative.

7. The DHM dosage powder of any one of claims 1 through 6, wherein the matrix material comprises hydroxypropyl methyl cellulose acetate succinate (HPMCAS).

8. The DHM dosage powder of any one of claims 1 through 7, wherein the matrix material comprises ethylcellulose and/or hydroxypropyl methylcellulose (HPMC).

9. The DHM dosage powder of any one of claims 1 through 8, wherein the matrix material comprises hydroxypropyl cellulose (HPC).

10. The DHM dosage powder of any one of claims 1 through 9, wherein the matrix material comprises a material selected from the group consisting of cellulose ester, cellulose acrylate, methylcellulose, hydroxyethyl cellulose, hydroxypropyl methyl cellulose propionate succinate, hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, carboxymethyl ethylcellulose (CMEC), hydroxypropyl methylcellulose acetate phthalate (HPMCAP), hydroxypropyl methylcellulose propionate phthalate, hydroxypropyl methylcellulose acetate trimellitate (HPMCAT), hydroxypropyl methylcellulose propionate trimellitate, cellulose acetate succinate (CAS), methyl cellulose acetate succinate (MCAS), sodium carboxymethylcellulose, and combinations.

11. The DHM dosage powder of any one of claims 1 through 10, wherein the matrix material comprises a material selected from the group consisting of polyvinyl pyrrolidone (PVP) and poly(vinyl pyrrolidone-co-vinyl acetate) (PVP-VA).

12. The DHM dosage powder of any one of claims 1 through 11, wherein the matrix material comprises polyethylene glycol (PEG).

13. The DHM dosage powder of any one of claims 1 through 12, wherein the matrix material comprises a material selected from the group consisting of a wax, polyoxyethylene-polyoxypropylene block copolymers (also referred to as poloxamers), polymethacrylates, polyoxyethylene alkyl ethers, polyoxyethylene castor oils, polycaprolactam, polycaprolactone (PCL), polylactic acid (PLA), polyglycolic acid (PGA), poly(lactic-glycolic acid) (PLGA), lipids, pullulan, dextran, hyaluronic acid, polysialic acid, chondroitin sulfate, heparin, fucoidan, pentosan polysulfate, spirulan, dextran, dextran acetate, dextran propionate, dextran succinate, dextran acetate propionate, dextran acetate succinate, dextran propionate succinate, dextran acetate propionate succinate, poly(methyl methacrylate) (PMMA), poly(methacrylic acid-co-methyl methacrylate) 1:1, poly(methacrylic acid-co-methyl methacrylate) 1:2, poly(methacrylic acid-co-ethyl acrylate) 1:1, a polysaccharide, polyethers, peptides, sugar oligomers, such as polydextrose and dextrans with molecular weights less than 10,000 Da, low molecular-weight oligomers of polyethylene glycols or poly amino acids or peptides, methacrylic acid copolymers, ethylene glycol-vinyl glycol copolymers, polyoxyl 40 hydrogenated castor oils, polymeric derivatives of vitamin E, N-methyl-2-pyrrolidone, cross linked polyvinyl N-pyrrolidone, a low melting point wax, such as carnauba wax, poly(propylene), poly(ethylene-co-vinyl acetate), starch, polyethoxylated sorbitan, polyoxyethylene sorbitan monooleate, polyethylene oxide, poly(ethylene oxide-co-vinyl acetate), poly(ethylene oxide-co-caprolactam), poly(ethylene oxide-co-vinyl acetate-co-caprolactam), and combinations.

14. The DHM dosage powder of any one of claims 1 through 13, wherein the matrix material comprises a material selected from the group consisting of a sugar, sugar alcohols, organic acids, salts of organic acids, fructose, glucose, lactose, mannitol, trehalose, sucrose, raffinose, maltitol, lactitol, sorbitol, xylitol, erythritol, xylose, acorbose, melezitose, galactose, melibrose, isomaltose, natural sugar extracts, malt beet sugar, corn sugar, high-fructose corn syrup, polyols, such as glycerol, sorbitol, ethylene glycol, propylene glycol, and butanediol, amino acids and salts of amino acids, such as glycine, leucine, serine, alanine, isoleucine, tri-leucine, organic acids and salts of organic acids, such as oleic acid, citric acid, tartaric acid, edetic acid, malic acid, sodium citrate, and combinations.

15. The DHM dosage powder of any one of claims 1 through 14, wherein the matrix material comprises an amphiphilic block copolymer.

16. The DHM dosage powder of claim 15, wherein the amphiphilic block copolymer is selected from the group consisting of polystyrene-block-polyethylene glycol (PS-b-PEG), polylactic acid-block-polyethylene glycol (PLA-b-PEG), and poly(lactic-co-glycolic acid)-block-polyethylene glycol (PLGA-b-PEG).

17. The DHM dosage powder of any one of claims 1 through 16, wherein the matrix material has a glass transition temperature (Tg) of at least 70° C., 100° C., or 115° C.

18. The DHM dosage powder of any one of claims 1 through 17, further comprising a permeabilizer.

19. The DHM dosage powder of claim 18, wherein the permeabilizer comprises capric acid, a caprate salt, and/or sodium caprate.

20. The DHM dosage powder of claim 18,
wherein the permeabilizer comprises a permeabilizer selected from the group consisting of a fatty acid, a saturated fatty acid, and/or a fatty acid complexed with a cation, a fatty acid, a fatty acid salt, a fatty acid metallic soap, and combinations and
wherein the cation is selected form the group consisting of a metal cation, a metal divalent cation, a magnesium divalent cation, a calcium divalent cation, a zinc divalent cation, an iron divalent cation, a metal trivalent cation, an iron trivalent cation, and combinations.

21. The DHM dosage powder of any one of claims 1 through 20, further comprising a coactive.

22. The DHM dosage powder of claim 21, wherein the coactive comprises an antioxidant.

23. The DHM dosage powder of any one of claims 21 and 22, wherein the coactive comprises glutathione.

24. The DHM dosage powder of any one of claims 21 through 23, wherein the coactive comprises L-cysteine.

25. The DHM dosage powder of any one of claims 21 through 24, wherein the coactive comprises a coactive selected from the group consisting of N-acetyl cysteine (NAC), Prickly Pear extract, Milk Thistle, Ginger Root, vitamin B, vitamin C, vitamin E, and combinations.

26. The DHM dosage powder of any one of claims 21 through 25, wherein the coactive comprises an electrolyte and/or a sugar.

27. The DHM dosage powder of any one of claims 1 through 26, further comprising a pH buffering agent.

28. The DHM dosage powder of claim 27, wherein the pH buffering agent is an acidic pH buffering agent.

29. The DHM dosage powder of claim 28, wherein the acidic pH buffering agent comprises citric acid, a citrate salt, a sodium citrate, a potassium citrate, calcium citrate, and/or combinations.

30. The DHM dosage powder of any one of claims 1 through 29, wherein the DHM is not solubilized or dissolved by an aqueous solution having a pH of at most 3.5.

31. The DHM dosage powder of any one of claims 1 through 29, wherein the DHM is not solubilized or dissolved by an aqueous solution having a pH of at most 2.

32. The DHM dosage powder of any one of claims 1 through 31, wherein the DHM is solubilized or dissolved by an aqueous solution having a pH of at least 5.5.

33. The DHM dosage powder of any one of claims 1 through 31, wherein the DHM is solubilized or dissolved by water or an aqueous solution having a pH of at least 7.

34. The DHM dosage powder of any one of claims 1 through 33, wherein the DHM comprises at least 5 wt % of the powder.

35. The DHM dosage powder of any one of claims 1 through 33, wherein the DHM comprises at least 20 wt % of the powder.

36. The DHM dosage powder of any one of claims 1 through 33, wherein the DHM comprises at least 40 wt % of the powder.

37. The DHM dosage powder of any one of claims 1 through 33, wherein the DHM comprises at least 55 wt % of the powder.

38. The DHM dosage powder of any one of claims 1 through 37, wherein the crystallinity of the DHM is at most 20%.

39. The DHM dosage powder of any one of claims 1 through 37, wherein the crystallinity of the DHM is at most 10%.

40. The DHM dosage powder of any one of claims 1 through 37, wherein the crystallinity of the DHM is at most 5%.

41. The DHM dosage powder of any one of claims 1 through 37, wherein the crystallinity of the DHM is at most 2%.

42. The DHM dosage powder of any one of claims 1 through 37, wherein the DHM is amorphous.

43. The DHM dosage powder of any one of claims 1 through 42, wherein the DHM dosage powder is molecularly dispersed.

44. The DHM dosage powder of any one of claims 1 through 42, wherein the DHM and the matrix material are homogeneously, molecularly, or uniformly dispersed in each other and the crystallinity of the DHM is at most 10%.

45. The DHM dosage powder of claim 44, wherein the crystallinity of the DHM is at most 5%.

46. The DHM dosage powder of claim 44, wherein the DHM is amorphous.

47. The DHM dosage powder of any one of claims 1 through 46, wherein the size of 80 wt % of particles in the powder is at least 5 μm.

48. The DHM dosage powder of any one of claims 1 through 46, wherein the size of 80 wt % of particles in the powder is at least 10 μm.

49. A dosage form, comprising
the DHM dosage powder of any one of claims 1 through 48, and
an enteric coating that encapsulates the DHM dosage powder.

50. The dosage form of claim 49, wherein the enteric coating is a polymeric coating.

51. The dosage form of claim 49, wherein the enteric coating is a methacrylate copolymer coating.

52. The dosage form of any one of claims 49 through 51, wherein the dosage form is a capsule, tablet, or pill.

53. A dosage form, comprising
the DHM dosage powder of any one of claims 1 through 48, and
an aqueous liquid,
wherein the DHM dosage powder is mixed with or suspended in the liquid.

54. A dosage form, comprising
the DHM dosage powder of any one of claims 1 through 48, and
a gel,
wherein the DHM dosage powder is mixed with or suspended in the gel.

55. The DHM dosage powder of claim 1,
wherein the matrix material comprises the polymer hydroxypropyl methyl cellulose acetate succinate (HPMCAS),
wherein the DHM comprises at least 10 wt % of the powder, and
wherein the crystallinity of the DHM is at most 10%.

56. The DHM dosage powder of claim 55, wherein the DHM and the matrix material are homogeneously or uniformly dispersed in each other.

57. The DHM dosage powder of claim 55, wherein the crystallinity of the DHM is at most 5%.

58. The DHM dosage powder of claim 57, wherein the DHM and the matrix material are homogeneously or uniformly dispersed in each other.

59. The DHM dosage powder of claim 55, wherein the DHM is amorphous.

60. The DHM dosage powder of claim 59, wherein the DHM and the matrix material are homogeneously or uniformly dispersed in each other.

61. The DHM dosage powder of claim 1,
wherein the matrix material is hydroxypropyl methyl cellulose acetate succinate (HPMCAS),
wherein the DHM comprises at least 20 wt % of the powder, and
wherein the crystallinity of the DHM is at most 10%.

62. The DHM dosage powder of claim 61, wherein the DHM and the matrix material are homogeneously or uniformly dispersed in each other.

63. The DHM dosage powder of claim 61, wherein the crystallinity of the DHM is at most 5%.

64. The DHM dosage powder of claim 63, wherein the DHM and the matrix material are homogeneously or uniformly dispersed in each other.

65. The DHM dosage powder of claim 61, wherein the DHM is amorphous.

66. The DHM dosage powder of claim 65, wherein the DHM and the matrix material are homogeneously or uniformly dispersed in each other.

67. The DHM dosage powder of claim 1,
wherein the matrix material is hydroxypropyl methyl cellulose acetate succinate (HPMCAS),
wherein the DHM comprises at least 40 wt % of the powder, and
wherein the crystallinity of the DHM is at most 10%.

68. The DHM dosage powder of claim 67, wherein the DHM and the matrix material are homogeneously or uniformly dispersed in each other.

69. The DHM dosage powder of claim 67, wherein the crystallinity of the DHM is at most 5%.

70. The DHM dosage powder of claim 69, wherein the DHM and the matrix material are homogeneously or uniformly dispersed in each other.

71. The DHM dosage powder of claim 67, wherein the DHM is amorphous.

72. The DHM dosage powder of claim 71, wherein the DHM and the matrix material are homogeneously or uniformly dispersed in each other.

73. The DHM dosage powder of any one of claims 55 through 72, so that mixing of the DHM dosage powder with a solvent results in a concentration of DHM dissolved in the solvent that is at least 20% greater than the equilibrium concentration of crystalline DHM dissolved in the solvent.

74. The DHM dosage powder of claim 73, wherein the solvent is an aqueous solvent.

75. The DHM dosage powder of claim 73, wherein the solvent is an aqueous solvent of pH in the range of 4.5 to 7.0.

76. The DHM dosage powder of claim 73, wherein the solvent is an aqueous solvent of pH in the range of 4.5 to 7.0 and comprising sodium at a concentration of from 100 mM to 400 mM.

77. The DHM dosage powder of claim 73, wherein the solvent is an aqueous solvent of pH in the range of 4.5 to 7.0 and comprising sodium at a concentration of from 100 mM to 400 mM and a surfactant at a concentration of from 0.01 wt % to 2 wt %.

78. The DHM dosage powder of claim 73, wherein the solvent is an aqueous solvent of pH in the range of 4.5 to 7.0 and comprising sodium at a concentration of from 100 mM to 400 mM and a nonionic surfactant at a concentration of from 0.05 wt % to 1.5 wt %.

79. The DHM dosage powder of claim 73, wherein the solvent is fed state simulated intestinal fluid (FeSSIF).

80. The DHM dosage powder of claim 73, wherein the solvent is fed state simulated intestinal fluid (FeSSIF) further comprising polysorbate at a concentration of about 1 wt %.

81. The DHM dosage powder of claim 73, wherein the solvent is fasted state simulated intestinal fluid (FaSSIF).

82. The DHM dosage powder of claim 73, wherein the solvent is fasted state simulated intestinal fluid (FaSSIF) further comprising polysorbate at a concentration of about 1 wt %.

83. The DHM dosage powder of any one of claims 55 through 72, so that mixing of the DHM dosage powder with a solvent results in a concentration of DHM dissolved in the solvent that is at least 50% greater than the equilibrium concentration of crystalline DHM dissolved in the solvent.

84. The DHM dosage powder of claim 83, wherein the solvent is an aqueous solvent.

85. The DHM dosage powder of claim 83, wherein the solvent is an aqueous solvent of pH in the range of 4.5 to 7.0.

86. The DHM dosage powder of any one of claim 83, wherein the solvent is an aqueous solvent of pH in the range of 4.5 to 7.0 and comprising sodium at a concentration of from 100 mM to 400 mM.

87. The DHM dosage powder of any one of claim 83, wherein the solvent is an aqueous solvent of pH in the range of 4.5 to 7.0 and comprising sodium at a concentration of from 100 mM to 400 mM and a nonionic surfactant at a concentration of from 0.01 wt % to 2 wt %.

88. The DHM dosage powder of claim 83, wherein the solvent is an aqueous solvent of pH in the range of 4.5 to 7.0 and comprising sodium at a concentration of from 100 mM to 400 mM and a polysorbate at a concentration of from 0.05 wt % to 1.5 wt %.

89. The DHM dosage powder of claim 83, wherein the solvent is fed state simulated intestinal fluid (FeSSIF).

90. The DHM dosage powder of claim 83, wherein the solvent is fed state simulated intestinal fluid (FeSSIF) further comprising polysorbate at a concentration of about 1 wt %.

91. The DHM dosage powder of claim 83, wherein the solvent is fasted state simulated intestinal fluid (FaSSIF).

92. The DHM dosage powder of claim 83, wherein the solvent is fasted state simulated intestinal fluid (FaSSIF) further comprising polysorbate at a concentration of about 1 wt %.

93. The DHM dosage powder of any one of claims 55 through 72, so that mixing of the DHM dosage powder with a solvent results in a concentration of DHM dissolved in the solvent that is at least 70% greater than the equilibrium concentration of crystalline DHM dissolved in the solvent.

94. The DHM dosage powder of claim 93, wherein the solvent is an aqueous solvent.

95. The DHM dosage powder of claim 93, wherein the solvent is an aqueous solvent of pH in the range of 4.5 to 7.0.

96. The DHM dosage powder of claim 93, wherein the solvent is an aqueous solvent of pH in the range of 4.5 to 7.0 and comprising sodium at a concentration of from 100 mM to 400 mM.

97. The DHM dosage powder of claim 93, wherein the solvent is an aqueous solvent of pH in the range of 4.5 to 7.0 and comprising sodium at a concentration of from 100 mM to 400 mM and a nonionic surfactant at a concentration of from 0.01 wt % to 2 wt %.

98. The DHM dosage powder of claim 93, wherein the solvent is an aqueous solvent of pH in the range of 4.5 to 7.0 and comprising sodium at a concentration of from 100 mM to 400 mM and a polysorbate at a concentration of from 0.05 wt % to 1.5 wt %.

99. The DHM dosage powder of claim 93, wherein the solvent is fed state simulated intestinal fluid (FeSSIF).

100. The DHM dosage powder of claim 93, wherein the solvent is fed state simulated intestinal fluid (FeSSIF) further comprising polysorbate at a concentration of about 1 wt %.

101. The DHM dosage powder of claim 93, wherein the solvent is fasted state simulated intestinal fluid (FaSSIF).

102. The DHM dosage powder of claim 93, wherein the solvent is fasted state simulated intestinal fluid (FaSSIF) further comprising polysorbate at a concentration of about 1 wt %.

103. The DHM dosage powder of any one of claims 55 through 72, wherein mixing of the DHM dosage powder with a solvent results in a concentration of DHM dissolved in the solvent that is at least 100% greater than the equilibrium concentration of crystalline DHM dissolved in the solvent.

104. The DHM dosage powder of claim 103, wherein the solvent is an aqueous solvent.

105. The DHM dosage powder of claim 103, wherein the solvent is an aqueous solvent of pH in the range of 4.5 to 7.0.

106. The DHM dosage powder of claim 103, wherein the solvent is an aqueous solvent of pH in the range of 4.5 to 7.0 and comprising sodium at a concentration of from 100 mM to 400 mM.

107. The DHM dosage powder of claim 103, wherein the solvent is an aqueous solvent of pH in the range of 4.5 to 7.0 and comprising sodium at a concentration of from 100 mM to 400 mM and a nonionic surfactant at a concentration of from 0.01 wt % to 2 wt %.

108. The DHM dosage powder of claim 103, wherein the solvent is an aqueous solvent of pH in the range of 4.5 to 7.0 and comprising sodium at a concentration of from 100 mM to 400 mM and a polysorbate at a concentration of from 0.05 wt % to 1.5 wt %.

109. The DHM dosage powder of claim 103, wherein the solvent is fed state simulated intestinal fluid (FeSSIF).

110. The DHM dosage powder of claim 103, wherein the solvent is fed state simulated intestinal fluid (FeSSIF) further comprising polysorbate at a concentration of about 1 wt %.

111. The DHM dosage powder of claim 103, wherein the solvent is fasted state simulated intestinal fluid (FaSSIF).

112. The DHM dosage powder of claim 103, wherein the solvent is fasted state simulated intestinal fluid (FaSSIF) further comprising polysorbate at a concentration of about 1 wt %.

113. A method for forming the dihydromyricetin (DHM) dosage powder of any one of claims 1 through 48 and claims 55 through 112 or the dosage form of any one of claims 49 through 54 by spray drying, comprising:
dissolving the dihydromyricetin (DHM) and the matrix material in a solvent to form a spray solution;
forcing the spray solution through an atomizer to atomize the spray solution; and
allowing the solvent to volatilize from the atomized spray solution to form the DHM dosage powder.

114. The method of claim 113, wherein the spray solution enters a drying chamber after being forced through the atomizer.

115. The method of any one of claims 113 and 114, wherein the volatilization of the solvent causes the polymer matrix material to gel without the DHM or matrix material phase separating from the atomized spray solution.

116. The method of any one of claims 113 through 115, wherein the solvent comprises acetone.

117. The method of any one of claims 113 through 116, wherein the solvent comprises tetrahydrofuran (THF).

118. The method of any one of claims 113 through 117, wherein the solvent comprises methanol, ethanol, and/or dimethylsulfoxide (DMSO).

119. The method of any one of claims 113 through 118, wherein the solvent comprises water.

120. The method of any one of claims 113 through 119, wherein the solvent comprises a solvent selected from the group consisting of an alcohol, n-propanol, iso-propanol, butanol, a ketones, acetone, methyl ethyl ketone (MEK), methyl iso-butyl ketone, an ester, ethyl acetate, propylacetate, acetonitrile, methylene chloride, toluene, a cyclic ether, 1,1,1-trichloroethane, a lower volatility solvent, dimethyl acetamido, and combinations.

121. The method of any one of claims 113 through 120, wherein a temperature of the drying chamber is at least 5° C., 10° C., or 20° C. greater than a boiling point temperature of the solvent.

122. A method for forming the dihydromyricetin (DHM) dosage powder of any one of claims 1 through 48 and claims 55 through 112 or the dosage form of any one of claims 49 through 54, comprising:
dissolving the dihydromyricetin (DHM) and hydroxypropylmethylcellulose acetate succinate (HPMCAS) as the matrix material in acetone at a total solids concentration of at least 2%, 5%, or 10% to form a spray solution,
wherein the weight ratio of DHM to HPMCAS is at least 5:95;
forcing the spray solution through an atomizer to atomize the spray solution; and
allowing the solvent to volatilize from the atomized spray solution to form the dihydromyricetin (DHM) dosage powder.

123. The method of claim 122, wherein the weight ratio of DHM to HPMCAS is at least 10:90.

124. The method of claim 122, wherein the weight ratio of DHM to HPMCAS is at least 20:80.

125. The method of claim 122, wherein the weight ratio of DHM to HPMCAS is at least 40:60.

126. A method for forming the dihydromyricetin (DHM) dosage powder of any one of claims 55 through 72, comprising:
dissolving the dihydromyricetin (DHM) and the hydroxypropylmethylcellulose acetate succinate (HPMCAS) in acetone at a total solids concentration of at least 10% to form a spray solution,
wherein the weight ratio of DHM to HPMCAS is at least 10:90;
forcing the spray solution through an atomizer to atomize the spray solution; and
allowing the solvent to volatilize from the atomized spray solution to form the dihydromyricetin (DHM) dosage powder.

127. The method of claim 126, wherein the weight ratio of DHM to HPMCAS is at least 20:80.

128. The method of claim 126, wherein the weight ratio of DHM to HPMCAS is at least 40:60.

129. The dihydromyricetin (DHM) dosage powder of any one of claims 1 through 48 and claims 55 through 112 or the dosage form of any one of claims 49 through 54 for use as a medicament.

130. The dihydromyricetin (DHM) dosage powder of any one of claims 1 through 48 and claims 55 through 112 or the dosage form of any one of claims 49 through 54 for use in reducing hangover symptoms.

131. The dihydromyricetin (DHM) dosage powder of any one of claims 1 through 48 and claims 55 through 112 or the dosage form of any one of claims 49 through 54 for use in preventing an alcohol use disorder.

132. The dihydromyricetin (DHM) dosage powder of any one of claims 1 through 48 and claims 55 through 112 or the dosage form of any one of claims 49 through 54 for use in preventing alcoholism.

133. The dihydromyricetin (DHM) dosage powder of any one of claims 1 through 48 and claims 55 through 112 or the dosage form of any one of claims 49 through 54 for use in treating an alcohol use disorder.

134. The dihydromyricetin (DHM) dosage powder of any one of claims 1 through 48 and claims 55 through 112 or the dosage form of any one of claims 49 through 54 for use in treating alcoholism.

135. The dihydromyricetin (DHM) dosage powder of any one of claims 1 through 48 and claims 55 through 112 or the dosage form of any one of claims 49 through 54 for use in treating an alcohol overdose.

136. The dihydromyricetin (DHM) dosage powder of any one of claims 1 through 48 and claims 55 through 112 or the dosage form of any one of claims 49 through 54 for use in increasing antioxidant capacity.

137. The dihydromyricetin (DHM) dosage powder of any one of claims 1 through 48 and claims 55 through 112 or the dosage form of any one of claims 49 through 54 for use in neuroprotection.

138. The dihydromyricetin (DHM) dosage powder of any one of claims 1 through 48 and claims 55 through 112 or the dosage form of any one of claims 49 through 54 for use in preventing Alzheimer's disease.

139. The dihydromyricetin (DHM) dosage powder of any one of claims 1 through 48 and claims 55 through 112 or the dosage form of any one of claims 49 through 54 for use in treating Alzheimer's disease.

140. The dihydromyricetin (DHM) dosage powder of any one of claims 1 through 48 and claims 55 through 112 or the dosage form of any one of claims 49 through 54 for use in inhibiting inflammation.

141. The dihydromyricetin (DHM) dosage powder of any one of claims 1 through 48 and claims 55 through 112 or the dosage form of any one of claims 49 through 54 for use in protection of the kidney.

142. The dihydromyricetin (DHM) dosage powder of any one of claims 1 through 48 and claims 55 through 112 or the dosage form of any one of claims 49 through 54 for use in protection of the liver.

143. The dihydromyricetin (DHM) dosage powder of any one of claims 1 through 48 and claims 55 through 112 or the dosage form of any one of claims 49 through 54 for use in preventing or treating cancer.

144. The dihydromyricetin (DHM) dosage powder of any one of claims 1 through 48 and claims 55 through 112 or the dosage form of any one of claims 49 through 54 for use in ameliorating a metabolic disorder.

145. The dihydromyricetin (DHM) dosage powder of any one of claims 1 through 48 and claims 55 through 112 or the dosage form of any one of claims 49 through 54 for use in preventing diabetes.

146. The dihydromyricetin (DHM) dosage powder of any one of claims 1 through 48 and claims 55 through 112 or the dosage form of any one of claims 49 through 54 for use in treating diabetes.

147. The dihydromyricetin (DHM) dosage powder of any one of claims 1 through 48 and claims 55 through 112 or the dosage form of any one of claims 49 through 54 for use in treating a bacterial infection.

148. Use of the dihydromyricetin (DHM) dosage powder of any one of claims 1 through 48 and claims 55 through 112 in the manufacture of a medicament for reducing hangover symptoms.

149. Use of the dihydromyricetin (DHM) dosage powder of any one of claims 1 through 48 and claims 55 through 112 in the manufacture of a medicament for preventing an alcohol use disorder, preventing alcoholism, treating an alcohol use disorder, treating alcoholism, and/or treating an alcohol overdose.

150. Use of the dihydromyricetin (DHM) dosage powder of any one of claims 1 through 48 and claims 55 through 112 in the manufacture of a medicament for neuroprotection, preventing Alzheimer's disease, and/or treating Alzheimer's disease.

151. Use of the dihydromyricetin (DHM) dosage powder of any one of claims 1 through 48 and claims 55 through 112 in the manufacture of a medicament for ameliorating a metabolic disorder, preventing diabetes, and/or treating diabetes.

152. Use of the dihydromyricetin (DHM) dosage powder of any one of claims 1 through 48 and claims 55 through 112 in the manufacture of a medicament for increasing antioxidant capacity, inhibiting inflammation, protecting the kidney, protecting the liver, preventing and/or treating cancer, and/or treating a bacterial infection.

153. A method for forming the dihydromyricetin (DHM) dosage powder of any one of claims 1 through 48 and claims 55 through 112 or the dosage form of any one of claims 49 through 54 by solvent evaporation, comprising:
dissolving the dihydromyricetin (DHM) and the matrix material in a solvent to form a solution; and
allowing the solvent to volatilize from the solution to form the DHM dosage powder.
